(19) United States
(12) Patent Application Publication
(10) Pub. No.: US 2017/0360409 A1
(43) Pub. Date: Dec. 21, 2017

(54) ULTRASOUND SIGNAL PROCESSING DEVICE, ULTRASOUND DIAGNOSTIC DEVICE, AND ULTRASOUND SIGNAL PROCESSING METHOD

(71) Applicant: Konica Minolta, Inc., Tokyo (JP)

(72) Inventor: Yasuaki SUSUMU, Osaka-shi (JP)

(21) Appl. No.: 15/617,572

(22) Filed: Jun. 8, 2017

(30) Foreign Application Priority Data
Jun. 16, 2016 (JP) 2016-120167

(51) Int. Cl. A61B 8/08; A61B 8/00; A61B 8/14

(52) U.S. Cl. CPC A61B 8/5207; A61B 8/5253; A61B 8/145; A61B 8/4444; A61B 8/4494

(57) ABSTRACT

An ultrasound signal processing device including: a transmitter performing transmission events while varying ultrasound beam travel direction; a reception processor generating an acoustic line signal for each transmission event that is performed by generating reception signal sequences and performing delay-and-summing; a combiner generating an intermediate combined acoustic line signal by combining acoustic line signals corresponding to the first to latest transmission events performed for a current frame; an evaluator judging whether a subsequent transmission event is to be performed for the current frame by calculating an evaluation value from an energy value of the intermediate combined acoustic line signal and judging whether the evaluation value satisfies a predetermined condition; and an outputter outputting the intermediate combined acoustic line signal as a combined acoustic line signal once the evaluator judges that a subsequent transmission event is not to be performed for the current frame.

This application is based on an application No. 2016-120167 filed on Jun. 16, 2016 in Japan, the contents of which are hereby incorporated by reference.

### BACKGROUND OF THE INVENTION

### (1) Field of the Invention

The present disclosure relates to an ultrasound signal processing device, an ultrasound diagnostic device including the ultrasound signal processing device, and an ultrasound signal processing method for the ultrasound signal processing device.

### (2) Description of the Related Art

There are two techniques for examining a subject using ultrasound. One is a technique using transmission beams whose wavefronts have shapes forming a focal point at one point or region of the subject (referred to as “focused ultrasound beams” in the following). The other is a technique using transmission beams whose wavefronts do not have shapes forming a focal point (referred to as “unfocused ultrasound beams” in the following).

One known examination technique deploying focused ultrasound beams is focus imaging (FI). Focus imaging achieves high spatial resolution around the focal point. Meanwhile, it is difficult to achieve high temporal resolution (frame rate) with focus imaging. This is because in focus imaging, spatial resolution is dependent upon distance from the focal point, and thus, an acoustic line signal covering only a small spatial range is produced through single transmission/reception of ultrasound (referred to as a “transmission event” in the following). Accordingly, focus imaging requires a large number of transmission events to generate a single ultrasound image.

Meanwhile, examination techniques deploying unfocused ultrasound beams include those deploying plane waves, those deploying diffused waves, and those deploying non-spherical waves. One known examination technique deploying unfocused ultrasound beams is plane wave imaging (PWI). In PWI, an acoustic line signal covering a large spatial range can be produced through a single transmission event due to the use of an unfocused ultrasound beam. Accordingly, PWI achieves high temporal resolution. Meanwhile, it is difficult to achieve high spatial resolution with PWI because unfocused ultrasound beams have lower spatial density compared to the focused ultrasound beams used in focus imaging. In view of this, a method referred to as the PWI synthetic aperture method is used to improve spatial resolution, as disclosed for example in Japanese Patent Publication No. 4114838.

In the PWI synthetic aperture method, transmission beam forming is performed to generate unfocused ultrasound beams with different travel directions. Further, a transmission event is performed for each unfocused ultrasound beam travel direction, and for each transmission event, an acoustic line signal is acquired by performing reception beam forming on receive signals acquired based on ultrasound reflection. Further, a combined acoustic line signal is generated by combining acoustic line signals corresponding to different unfocused ultrasound beam travel directions.

### SUMMARY OF THE INVENTION

### Problems to be Solved by the Invention

With the PWI synthetic aperture method, the larger the number of acoustic line signals combined to generate a combined acoustic line signal (i.e., the larger the number of transmission events performed), typically the higher the spatial resolution of the combined acoustic line signal. This means that a large number of transmission events need to be performed to generate one ultrasound image. Thus, it is conventionally difficult to achieve high temporal resolution with the PWI synthetic aperture method.

The present invention has been made in view of such technical problems, and an aim thereof is to provide an ultrasound signal processing device and an ultrasound diagnostic device including the ultrasound signal processing device that adaptively achieve both high spatial resolution and high temporal resolution in a synthetic aperture method using unfocused ultrasound beams.

### Means for Solving the Problems

One aspect of the present invention is an ultrasound signal processing device that performs multiple transmission events of transmitting non-focused ultrasound beams to a subject by using an ultrasound probe having multiple transducers, that performs, for each of the transmission events, reception of ultrasound reflection from the subject and generation of an acoustic line signal based on the ultrasound reflection, and that combines acoustic line signals for the respective transmission events to generate a combined acoustic line signal, the ultrasound signal processing device including ultrasound signal processing circuitry configured to operate as: a transmitter that varies ultrasound beam travel direction between transmission events and performs each transmission event by causing the ultrasound probe to transmit an ultrasound beam traveling to an inside of the subject; a reception processor that generates an acoustic line signal for each transmission event that is performed by generating reception signal sequences for transducers based on ultrasound reflection that the ultrasound probe receives from a target region inside the subject and performing delay-and-summing using the reception signal sequences; a combiner that generates an intermediate combined acoustic line signal by combining, based on measurement points in the target region, acoustic line signals corresponding to the first to latest transmission events performed for a current combined acoustic line signal; an evaluator that judges whether a subsequent transmission event is to be performed for the current combined acoustic line signal by calculating an evaluation value from an energy value of the intermediate combined acoustic line signal and judging whether the evaluation value satisfies a predetermined condition; and an outputter that outputs the intermediate combined acoustic line signal as the current combined acoustic line signal once the evaluator judges that a subsequent transmission event is not to be performed for the current combined acoustic line signal.

### Advantageous Effect of the Invention

The ultrasound signal processing device pertaining to one aspect of the present invention, as well as an ultrasound diagnostic device including the ultrasound signal processing device, adaptively change the number of transmission events performed based on a relation between the number of acoustic line signals that are combined to generate a combined acoustic line signal and the quality of the combined acoustic line signal. As such, the ultrasound signal processing device pertaining to one aspect of the present invention and the ultrasound diagnostic device including the ultrasound signal processing device achieve both high spatial resolution and high temporal resolution by suppressing the number of transmission events performed to as small as possible while keeping spatial resolution at a certain level or higher.

### BRIEF DESCRIPTION OF THE DRAWINGS

These and the other objects, advantages and features of the invention will become apparent from the following description thereof taken in conjunction with the accompanying drawings, which illustrate specific embodiment(s) of the invention.

In the drawings:

FIG. 1 is a functional block diagram illustrating an ultrasound diagnostic system 1000 pertaining to embodiment 1;

FIG. 2 is a functional block diagram illustrating an ultrasound signal processing device 110 pertaining to embodiment 1;

FIG. 3 is a flowchart illustrating operations of an ultrasound diagnostic device 100 pertaining to embodiment 1;

FIG. 4 is a flowchart illustrating operations of an evaluator 155 pertaining to embodiment 1;

FIGS. 5A through 5C are each a schematic illustrating a relation between evaluation value and transmission count;

FIG. 6 is a schematic illustrating a relation between ultrasound beam travel direction and blank region;

FIG. 7 is a flowchart illustrating operations of an evaluator pertaining to modification 1;

FIG. 8 is a flowchart illustrating operations of an evaluator pertaining to modification 2;

FIG. 9 is a functional block diagram illustrating an ultrasound signal processing device 210 pertaining to embodiment 2;

FIG. 10 is a flowchart illustrating operations of an ultrasound diagnostic device pertaining to embodiment 2;

FIG. 11 is a flowchart illustrating a fixed transmission count determination operation pertaining to embodiment 2;

FIG. 12 is a flowchart illustrating an image acquisition operation pertaining to embodiment 2;

FIG. 13 is a flowchart illustrating operations of an ultrasound diagnostic device pertaining to modification 3;

FIGS. 14A through 14F are each a timing chart illustrating a relation between frame rate and operations; and

FIG. 15 is a schematic illustrating a relation between combined acoustic line signal pulse width and the number of transmission events.

### DESCRIPTION OF EMBODIMENTS

### <Process Leading to Embodiments of Invention>

The inventor conducted various observations in order to achieve both high spatial resolution and high temporal resolution in an ultrasound signal processing device and an ultrasound diagnostic device deploying a synthetic aperture method.

In the PWI synthetic aperture method, multiple plane waves with different travel directions are transmitted, acoustic line signals are generated based on ultrasound reflection, one for each plane wave, and the acoustic line signals so generated are combined to generate a combined acoustic line signal. This achieves the two effects described in the following. First, the combined acoustic line signal has higher spatial resolution than the individual acoustic line signals. Specifically, the acoustic line signals that are combined each correspond to a different ultrasound travel direction, and thus have different orientations from one another in terms of both directional resolution and distance resolution. Accordingly, the combined acoustic line signal, generated by combining these acoustic line signals, has higher spatial resolution in two dimensions than the original acoustic line signals. Secondly, the combined acoustic line signal maintains high spatial resolution even if there is anisotropy inside the measurement-target subject. Specifically, when the measurement-target subject includes a structure that strongly reflects ultrasound, individual acoustic line signals unfortunately tend to have low quality due to low intensity of ultrasound reflection. This is because plane waves are not capable of sufficiently reaching a region that is shaded by the structure, or more specifically, a region near the subject and located in the ultrasound travel direction from the structure. However, when transmitting multiple plane waves with different travel directions and generating an acoustic line signal for each plane wave based on ultrasound reflection, the region that is shaded by the structure would differ between the acoustic line signals. Specifically, a region that is shaded from a plane wave for one acoustic line signal by the structure receives a plane wave for a different acoustic line signal without being shaded by the structure. Accordingly, the combined acoustic line signal generated by combining these acoustic line signals maintains high spatial resolution even if there is anisotropy inside the measurement-target subject.

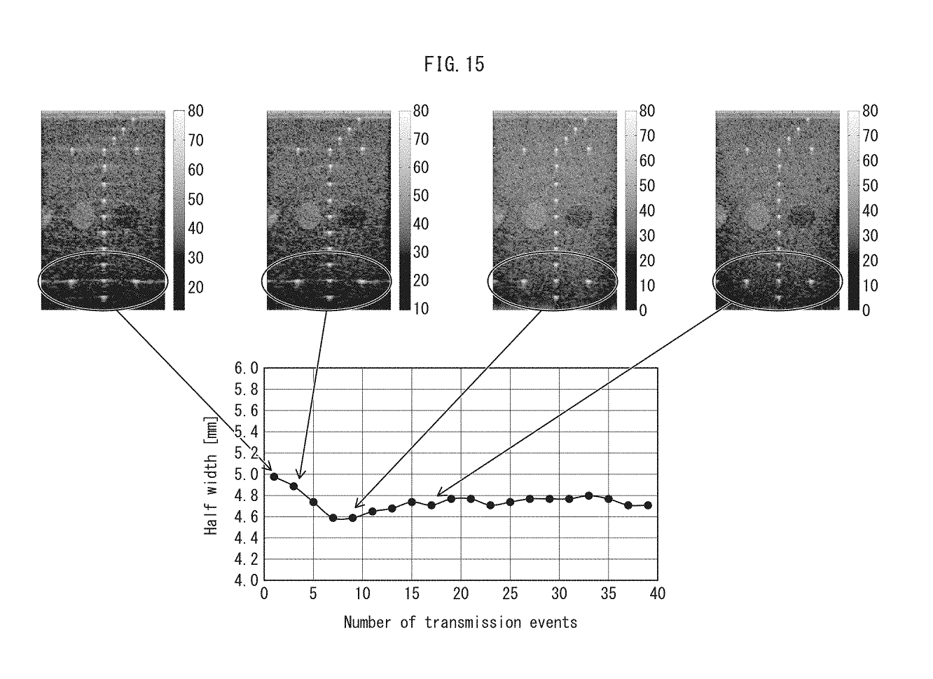

Meanwhile, it is not necessarily preferable to perform a large number of transmission events to generate a single ultrasound image, as disclosed in Japanese Patent Application Publication No. 2006-51355. This is for the following reasons. The first reason is that performing a large number of transmission events results in a long amount of time being required for generating a single ultrasound image, and thus, results in difficulty in achieving high temporal resolution (frame rate). This may further lead to deterioration of real-time performance of an ultrasound diagnostic device. Further, performing a large number of transmission events may even result in a decrease in spatial resolution, particularly when an ultrasound probe, a tissue inside the subject, etc., moves. Specifically, when an ultrasound probe, a tissue, etc., moves while the acoustic line signals to be combined to generate the combined acoustic line signal are being acquired, an imaging-target object may have different states in the acoustic line signals. The second reason is that spatial resolution does not necessarily increase in proportion to the increase in the number of transmission events performed. Instead, the contribution that a single acoustic line signal makes in improving combined acoustic line signal spatial resolution is substantially inversely-proportional to the number of acoustic line signals that are combined to generate the combined acoustic line signal. This means that in order to continue increasing combined acoustic line signal spatial resolution at the same rate, the number of acoustic line signals that need to be combined increases exponentially. That is, the degree of improvement in spatial resolution would become smaller as the total number of acoustic line signals used to generate the combined acoustic line signal increases, even when increasing the number of acoustic line signals that are combined at a same rate. FIG. 15 illustrates a relation between the number of transmission events and the spatial resolution of the combined acoustic line signal (half width (full width at half maximum, or half width at half maximum) of combined acoustic line signal pulse). FIG. 15 illustrates that combined acoustic line signal spatial resolution is greatest (half width becomes smallest) when the number of transmission events performed is around six to eight, and spatial resolution rather decreases without increasing when increasing the number of transmission events beyond this range. This situation is considered to occur for the following reason. Once the number of acoustic line signals combined exceeds five or a similar value, improvement in spatial resolution can no longer be expected even if the number of acoustic line signals combined is further increased. Instead, a decrease in spatial resolution can be expected because movement of an ultrasound probe, etc., would occur when the amount of time spent for ultrasound transmission/reception increases.

In view of such technical problems, the inventor considered a technique of reducing the number of transmission events performed for generating a combined acoustic line signal within a range ensuring that the combined acoustic line signal has sufficient spatial resolution, and thereby arrived at the ultrasound signal processing devices, the ultrasound diagnostic devices, and the ultrasound signal processing methods pertaining to the embodiments.

The following provides detailed description of the ultrasound signal processing devices, the ultrasound diagnostic devices, and the ultrasound signal processing methods pertaining to the embodiments, with reference to the accompanying drawings.

### Embodiment 1

### <Overall Structure>

The following describes an ultrasound diagnostic device 100 pertaining to embodiment 1, with reference to the accompanying drawings.

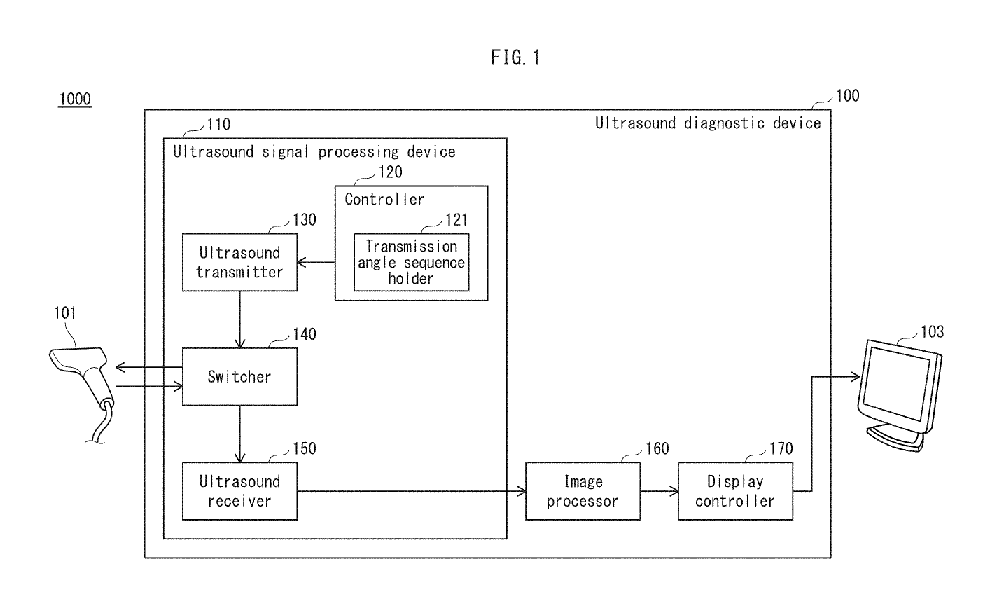

FIG. 1 is a functional block diagram illustrating an ultrasound diagnostic system 1000 pertaining to embodiment 1. As illustrated in FIG. 1, the ultrasound diagnostic system 1000 includes an ultrasound probe 101 and a display 103, in addition to, the ultrasound diagnostic device 100. The ultrasound probe 101 has transducers 101a that transmit ultrasound towards a measurement-target subject and receive ultrasound reflection from the measurement-target subject. The ultrasound diagnostic device 100 causes the ultrasound probe 101 to transmit and receive ultrasound and generates an ultrasound image based on output signals from the ultrasound probe 101. The display 103 displays the ultrasound image on a screen. The ultrasound probe 101 and the display 103 are each configured to be connectable to the ultrasound diagnostic device 100, and FIG. 1 illustrates the ultrasound diagnostic device 100 with the ultrasound probe 101 and the display 103 connected thereto. Alternatively, the ultrasound probe 101 and the display 103 may be included in the ultrasound diagnostic device 100.

### <Structure of Ultrasound Diagnostic Device 100>

The ultrasound diagnostic device 100 includes a switcher 140, an ultrasound transmitter 130, an ultrasound receiver 150, and a controller 120. The switcher 140 selects transducers 101a to be used for transmission (referred to in the following as transmit transducers 101a) and transducers 101a to be used for reception (referred to in the following as receive transducers 101a), from among the transducers 101a of the ultrasound probe 101, and secures input to and output from the selected transducers 101a. The ultrasound transmitter 130 generates element drive signals for the transmit transducers 101a to achieve ultrasound transmission. Meanwhile, the ultrasound receiver 150 performs reception processing by using ultrasound reflection received by the ultrasound probe 101. The controller 120 includes a transmission angle sequence holder 121, and controls the ultrasound transmitter 130 and the ultrasound receiver 150. The ultrasound diagnostic device 100 further includes an image processor 160 and a display controller 170. The image processor 160 generates a tomographic image (a B-mode image) based on a combined acoustic line signal generated by the ultrasound receiver 150. The display controller 170 causes the display 103 to display the tomographic image generated by the image processor 160.

Among such constituent elements, the switcher 140, the ultrasound transmitter 130, the ultrasound receiver 150, and the controller 120 form an ultrasound signal processing device 110, which constitutes ultrasound signal processing circuitry.

The constituent elements of the ultrasound diagnostic device 100, for example the switcher 140, the ultrasound transmitter 130, the ultrasound receiver 150, the controller 120, the image processor 160, and the display controller 170, are each implemented by using a hardware circuit such as a field programmable gate array (FPGA) or an application specific integrated circuit (ASIC).

The ultrasound diagnostic device 100 pertaining to the present embodiment is not limited to having the structure illustrated in FIG. 1. For example, the ultrasound diagnostic device 100 may not include the switcher 140, in which case the ultrasound transmitter 130 and the ultrasound receiver 150 may be directly connected to transducers 101a. Further, the ultrasound probe 101 may include, as built-in components, the ultrasound transmitter 130 and the ultrasound receiver 150, or may include some of the components included in the ultrasound transmitter 130 and the ultrasound receiver 150. The above not only applies to the ultrasound diagnostic device 100 pertaining to the present embodiment, but also similarly applies to ultrasound diagnostic devices pertaining to the rest of the embodiments and modifications that are described later in the present disclosure.

### <Ultrasound Signal Processing Device 110>

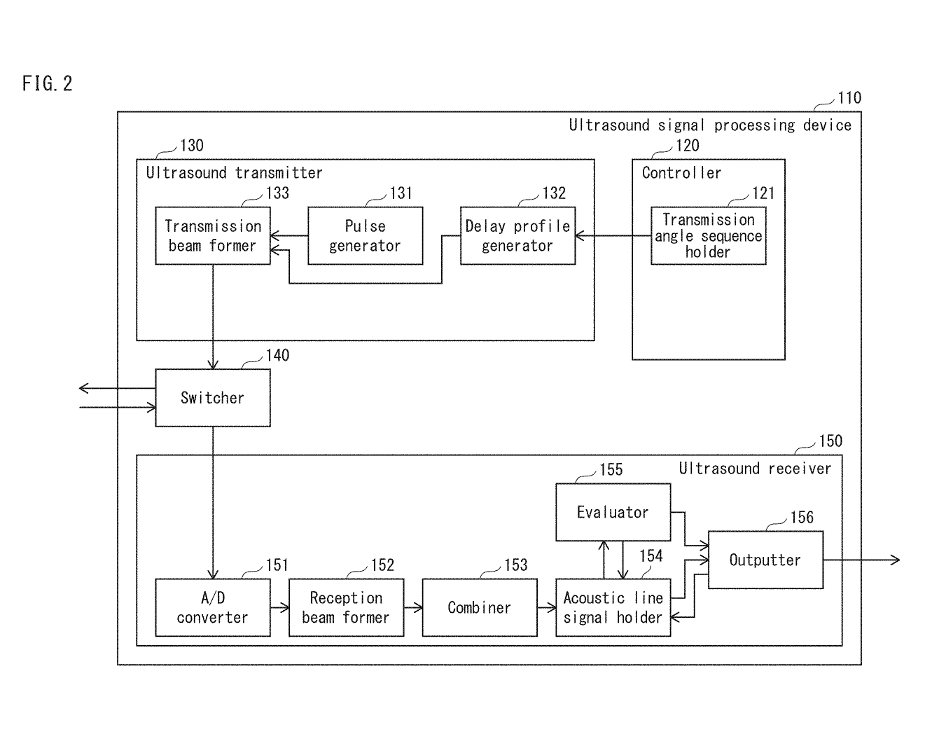

FIG. 2 is a detailed functional block diagram of the ultrasound signal processing device 110 pertaining to embodiment 1.

### 1. Ultrasound Transmitter 130

The switcher 140 connects the ultrasound transmitter 130 to the ultrasound probe 101. The ultrasound transmitter 130 controls timings when high voltages causing ultrasound transmission from the ultrasound probe 101 are applied to the transmit transducers 101a. The ultrasound transmitter 130 includes a pulse generator 131, a delay profile generator 132, and a transmission beam former 133.

The pulse generator 131, based on a transmission control signal from the controller 120, performs transmission processing of supplying a pulsed transmission signal for causing the transmit transducers 101a to transmit an ultrasound beam. That is, the pulse generator 131 is a pulse generation circuit for generating a pulsed signal for driving the transmit transducers 101a.

The delay profile generator 132 acquires a transmission angle sequence from the transmission angle sequence holder 121, and generates a delay profile defining delay amounts for the transmit transducers 101a. The delay profile is generated so that an ultrasound beam transmitted from the ultrasound probe 101 travels in a transmission angle defined in the transmission angle sequence. Specifically, the delay profile generator 132 first acquires the transmission angle sequence from the transmission angle sequence holder 121. Here, the transmission angle sequence is data of a numerical sequence including transmission angles arranged in the order they are to be used. Each transmission angle indicates an angle of an ultrasound beam travel direction relative to a normal direction of a direction in which the transducers 101a form a line (referred to in the following as an element line direction). For example, the transmission angle sequence may be {0, −0.2, +0.2, −0.4, +0.4, . . . }. Here, supposing that the transducers 101a form a line along a left-right direction, a positive or negative sign provided to a transmission angle indicates whether the corresponding ultrasound beam travel direction is inclined to the right or to the left. Further, a difference between two transmission angles corresponds to an angle between two ultrasound beam travel directions indicated by the respective transmission angles. For example, when supposing that 0° indicates a top-to-bottom direction, that a positive sign indicates inclination to the right, and that a negative sign indicates inclination to the left, an ultrasound beam with a −20° transmission angle travels towards the bottom left, and an ultrasound beam with a +20° transmission angle travels towards the bottom right. The delay profile generator 132 extracts the transmission angles in the transmission angle sequence one by one, and based on each transmission angle, generates a delay profile defining delay amounts for the transmit transducers 101a.

The transmission beam former 133 generates element drive signals for driving the respective transmit transducers 101a based on the transmission signal generated by the pulse generator 131 and the delay profile generated by the delay profile generator 132. Specifically, the transmission beam former 133 includes, for example, a clock generation circuit and a delay circuit. The clock generation circuit generates a clock signal that determines a transmission timing of an ultrasound beam. The transmission beam former 133 generates element drive signals for the respective transmit transducers 101a by using the delay circuit and delaying the transmission signal in accordance with the delay profile. Accordingly, the ultrasound probe 101 emits an ultrasound beam whose travel direction is in accordance with a transmission angle included in the transmission angle sequence.

### 2. Structure of Ultrasound Receiver 150

The ultrasound receiver 150 generates an acoustic line signal from electric signals that are acquired by receive transducers 101a based on ultrasound reflection received by the ultrasound probe 101. In the present disclosure, an acoustic line signal is a signal including delay-and-summed signals for measurement points in a target region. The ultrasound receiver 150 includes an A/D converter 151, a reception beam former 152, a combiner 153, an acoustic line signal holder 154, an evaluator 155, and an outputter 156.

The following describes the structures of the constituent elements of the ultrasound receiver 150.

### (1) A/D Converter 151

The switcher 140 connects the A/D converter 151 to the ultrasound probe 101. For each transmission event, the A/D converter 151 generates element receive signals (radio-frequency (RF) signals). The element receive signals are acquired by amplifying electric signals acquired through reception of ultrasound reflection by the ultrasound probe 101, and then performing analog-to-digital (A/D) conversion. The A/D converter 151 performs the generation of element receive signals and the outputting of the receive signals to the reception beam former 151 for multiple transmission events one after another.

Here, an element receive signal (an RF signal) is a digital signal acquired by amplifying an electric signal converted from ultrasound reflection received by a receive transducer 101a and then performing A/D conversion. An element receive signal for a receive transducer 101a is a sequence of signal components along an ultrasound transmission direction (subject depth direction) received by the receive transducer 101a.

### (2) Reception Beam Former 152

The reception beam former 152 is a circuit that generates an acoustic line signal for each transmission event. Specifically, the reception beam former 152, for each measurement point Pⱼₖ in the target region, performs delay-and-summing of element receive signals received by the receive transducers from the measurement point Pⱼₖ. Further, the reception beam former 152 combines the delayed-and-summed signals for the measurement points Pⱼₖ to generate an acoustic line signal. In the present disclosure, the target region is a region in which the measurement points Pⱼₖ exist, and is set by the controller 120. For example, the target region is a rectangular region defined based on the width of the ultrasound probe 101 and a predetermined depth.

### (3) Combiner 153

The combiner 153 performs combining of acoustic line signals. The number of acoustic line signals that the combiner 153 uses in the combining equals the number of transmission events having been performed. Specifically, the combiner 153 acquires an acoustic line signal from the reception beam former 152, and then combines the acoustic line signal with an intermediate combined acoustic line signal stored in the acoustic line signal holder 154. The intermediate combined acoustic line signal stored in the acoustic line signal holder 154 is a signal that the combiner 153 has previously generated by performing the combining. More specifically, the combiner 153 first reads out an intermediate combined acoustic line signal and a combination count C from the acoustic line signal holder 154. The combination count C indicates the number of acoustic line signals from which the intermediate combined acoustic line signal stored in the acoustic line signal holder 154 has been generated. Subsequently, the combiner 153, for each measurement point Pⱼₖ, multiples value VSⱼₖ for the measurement point Pⱼₖ in the acquired intermediate combined acoustic line signal and the combination count C, and then adds value Vⱼₖ for the measurement point Pⱼₖ in the acoustic line signal acquired from the reception beam former 152. Finally, the combiner 153 divides the calculated value (C×VSⱼₖ+Vⱼₖ) for each measurement point Pⱼₖ by (C+1) to generate a new intermediate combined acoustic line signal, and increments the combination count C (C=C+1). Note that when the combination count C acquired from the acoustic line signal holder 154 is zero, the combiner 153 may increment the combination count C to one and store the acoustic line signal acquired from the reception beam former 152 as-is as a new intermediate combined acoustic line signal to the acoustic line signal holder 154. Through this combining, a new intermediate combined acoustic line signal is generated that includes, for each measurement point Pⱼₖ, an average of values for the measurement point Pⱼₖ in one or more acoustic line signals from an acoustic line signal corresponding to the initial transmission event to the acoustic line signal corresponding to the latest transmission event. Note that a modification may be made such that the combiner 153 stores both an intermediate combined acoustic line signal and acoustic line signals to the acoustic line signal holder 154, and generates a new intermediate combined acoustic line signal by reading out all acoustic line signals already stored to the acoustic line signal holder 154 and by combining these acoustic line signals, instead of reading an intermediate combined acoustic line signal and the combination count C from the acoustic line signal holder 154. Further, the combiner 153 stores the new intermediate combined acoustic line signal that it has generated and the incremented combination count C to the acoustic line signal holder 154.

### (4) Acoustic Line Signal Holder 154

The acoustic line signal holder 154 is a storage medium that stores the combination count C and an intermediate combined acoustic line signal that the combiner 153 has generated by combining one or more acoustic line signals from the acoustic line signal corresponding to the initial transmission event to the acoustic line signal corresponding to the latest transmission event. In addition, the acoustic line signal holder 154 stores energy values. For example, the acoustic line signal holder 154 is implemented by using a hard disk, an optical disc, or a semiconductor storage device such as a flash memory.

### (5) Evaluator 155

The evaluator 155 judges whether the intermediate combined acoustic line signal stored in the acoustic line signal holder 154 satisfies a predetermined condition. Specifically, the evaluator 155 calculates an evaluation value based on the intermediate combined acoustic line signal, and judges whether the evaluation value is lower than a threshold value.

The evaluation value is calculated based on an energy value E of the intermediate combined acoustic line signal stored in the acoustic line signal holder 154. The following describes how the energy value E is calculated, and then how the evaluation value is calculated.

First, the evaluator 155 acquires the intermediate combined acoustic line signal stored in the acoustic line signal holder 154. Then, the evaluator 155 sets a region of interest (ROI) from which the energy value Eᵢ is to be calculated. Here, description is provided supposing that the entire target region is the ROI. Subsequently, the evaluator 155 calculates, for each measurement point Pⱼₖ in the ROI, an amplitude value Aⱼₖ of the intermediate combined acoustic line signal at the measurement point Pⱼₖ. Further, the evaluator 155 sets a sum of absolute values of amplitude values Aⱼₖ for all measurement points Pⱼₖ as the energy value Eᵢ. The evaluator 155 stores the energy value E so calculated to the acoustic line signal holder 154. Note that instead of using absolute values of amplitude values Aⱼₖ of the intermediate combined acoustic line signal, the evaluator 155 may for example use squares of amplitude values Aⱼₖ of the intermediate combined acoustic line signal.

The following describes how the evaluator 155 calculates the evaluation value. The evaluator 155 acquires an energy value EA pertaining to a previous transmission event and an energy value Eᵢ₋₁ pertaining to the initial transmission event. Then, the evaluator 155 calculates the evaluation value (evaluation value Kᵢ) for the intermediate combined acoustic line signal by using the following expression.

Kᵢ=(Eᵢ−Eᵢ₋₁)/E₁

That is, the evaluation value Kᵢ indicates contribution of an i^{th} acoustic line signal (i.e., acoustic line signal corresponding to an i^{th} transmission event) to an i^{th} intermediate combined acoustic line signal in terms of energy, when regarding that the energy value E₁ pertaining to the initial transmission event is 1.0.

Finally, the evaluator 155 determines whether the evaluation value Kᵢ so calculated is lower than the threshold value. The threshold value is preferably around 0.7 to 0.9 for example. In the following, description is provided supposing that the threshold value is 0.8. The evaluator 155, when the evaluation value Kᵢ is lower than this threshold value, provides an instruction to the outputter 156 to output the intermediate combined acoustic line signal stored in the acoustic line signal holder 154 as a combined acoustic line signal.

### (6) Outputter 156

Once the evaluator 155 judges that the intermediate combined acoustic line signal stored in the acoustic line signal holder 154 satisfies the predetermined condition, the outputter 156 outputs the intermediate combined acoustic line signal as a combined acoustic line signal, and deletes the intermediate combined acoustic line signal and the combination count C stored in the acoustic line signal holder 154. Alternatively, the outputter 156 may delete only the combination count C.

### 3. Controller 120

The controller 120 includes the transmission angle sequence holder 121. The controller 120, while changing the ultrasound beam travel direction, causes the ultrasound transmitter 130 to perform transmission of ultrasound beams and causes the ultrasound receiver 150 to perform reception processing of ultrasound reflection generated by the ultrasound beams. Specifically, the controller 120 performs the following operations. The controller 120 reads out the transmission angle sequence from the transmission angle sequence holder 121. Subsequently, the controller 120 causes the ultrasound transmitter 130 to transmit an ultrasound beam based on the first transmission angle, and then causes the ultrasound receiver 150 to perform reception processing of ultrasound reflection generated by the ultrasound beam. Subsequently, unless receiving a signal indicating completion of image acquisition for a current frame from the ultrasound receiver 150, the controller 120 causes the ultrasound transmitter 130 to transmit an ultrasound beam based on the second transmission angle, and then causes the ultrasound receiver 150 to perform reception processing of ultrasound reflection generated by the ultrasound beam. Following this, the controller 120 causes ultrasound beam transmission/reception based on subsequent transmission angles until it receives the signal from the ultrasound receiver 150. Meanwhile, when receiving the signal from the ultrasound receiver 150, the controller 120 regards that the transmission event having been performed most recently is the final transmission event for the current frame. Subsequently, the controller 120 causes an initial transmission event for generating a combined acoustic line signal for a subsequent frame to be commenced, based on a predetermined frame rate.

### 4. Switcher 140

The switcher 140 is a circuit that connects the ultrasound transmitter 130 to the transmit transducers 101a, and connects the ultrasound receiver 150 to the receive transducers 101a. Specifically, upon transmission of an ultrasound beam, the switcher 140 connects the ultrasound transmitter 130 and the transmit transducers 101a so that the element drive signals from the ultrasound transmitter 130 are each supplied to a corresponding transmit transducer 101a. Further, upon reception processing, the switcher 140 connects the ultrasound receiver 150 and receive transducers 101a.

### <Structures of Other Constituent Elements of Ultrasound Diagnostic Device 100>

The image processor 160 is a circuit converting a combined acoustic line signal output from the outputter 156 of the ultrasound receiver 150 into a B-mode image signal, by performing processing such as conversion into coordinates on an orthogonal coordinate system, and conversion into luminance signals through envelope detection and logarithmic compression.

The display controller 170 is a circuit causing the display 103 to display the latest B-mode image generated by the image processor 160.

### <Operations>

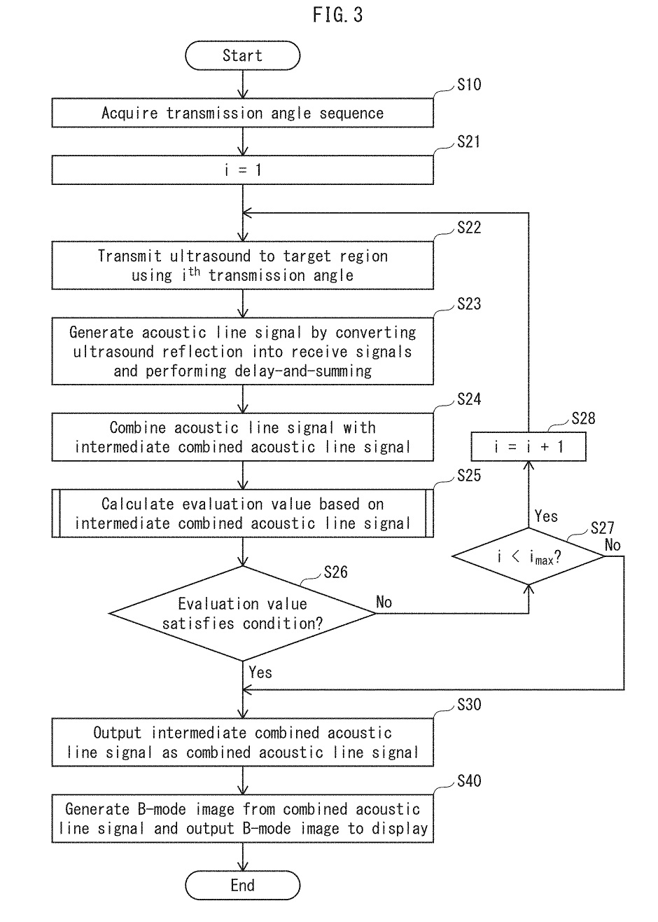

The following describes operations of the ultrasound diagnostic device 100 with the above-described structure. FIG. 3 is a flowchart illustrating the operations of the ultrasound diagnostic device 100.

First, the ultrasound transmitter 130 acquires a transmission angle sequence from the transmission angle sequence holder 121 in Step S10. Description is provided in the following supposing that the acquired transmission angle sequence is {0, −5, +5, −10, +10, −15, +15, . . . , −40, +40}.

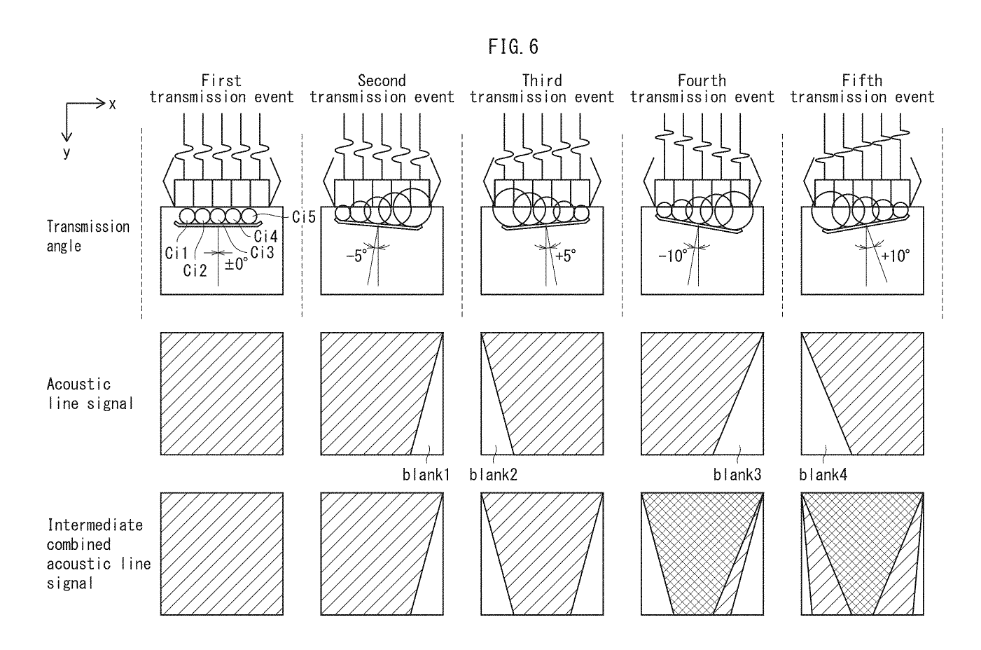

Subsequently, one is set to a counter that measures a transmission count i (i.e., the counter is initialized) (Step S21), and an ultrasound beam is transmitted to the target region by using the i^{th} transmission angle (Step S22). Here, since i equals one, the first transmission angle, namely 0°, is used as the transmission angle. Further, the ultrasound transmitter 130 generates element drive signals driving all transmit transducers 101a at the same time as illustrated in FIG. 6, and the ultrasound probe 101 transmits an ultrasound beam in a direction perpendicular to the element line direction. That is, an ultrasound beam with a wavefront parallel to the element line direction is transmitted.

In subsequent Step S23, ultrasound reflection generated by the transmitted ultrasound beam is converted into element receive signals, and an acoustic line signal is generated through delay-and-summing of the element receive signals. Specifically, the ultrasound beam is reflected inside the subject and arrives at the ultrasound probe 101 as ultrasound reflection. Accordingly, first, the receive transducers 101a convert the ultrasound reflection into electric signals, and output the electric signals to the ultrasound receiver 150. Then, the A/D converter 151 converts the electric signals into element receive signals (RF signals) by performing amplification and A/D conversion of the electric signals. Subsequently, the reception beam former 152 generates an acoustic line signal (DAS data) by performing, for each measurement point Pⱼₖ in the target region, delaying of the element receive signals to align timings of signal components of the element receive signals corresponding to ultrasound reflection received from the measurement point Pⱼₖ, and then performing summing of the delayed receive signals. Here, the target region is a region for which an acoustic line signal is to be generated, and description in the present embodiment is provided supposing that the target region is set in advance.

In subsequent Step S24, an intermediate combined acoustic line signal is generated through the combining of acoustic line signals. The combiner 153 combines all acoustic line signals corresponding to values of i from one to the current value. Since i equals one here, the combining is performed using only the acoustic line signal generated in the immediately-preceding Step S23. Thus, the combiner 153 stores the acoustic line signal generated in Step S23 as-is to the acoustic line signal holder 154 as an intermediate combined acoustic line signal.

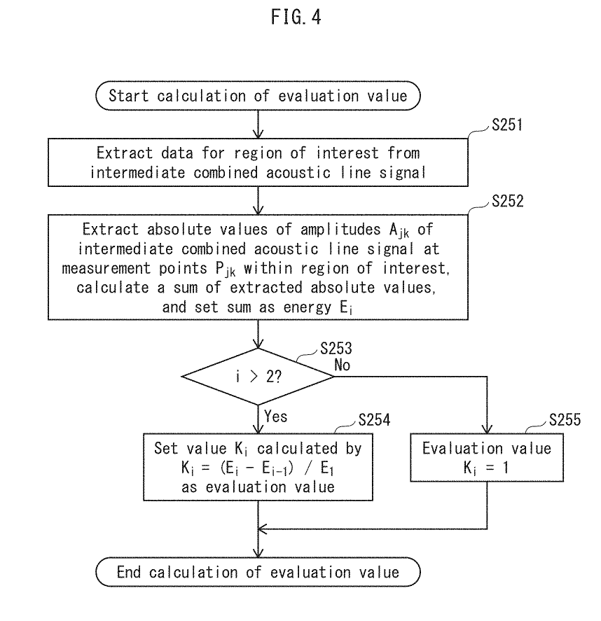

In subsequent Step S25, an evaluation value is calculated based on the intermediate combined acoustic line signal stored in the acoustic line signal holder 154. FIG. 4 is a flowchart illustrating a calculation process of the evaluation value.

First, in Step S251, data for a predetermined ROI is extracted from the intermediate combined acoustic line signal. Here, the ROI is a region for which an evaluation value is to be calculated, and is either a part of the target region or the entire target region. In the present embodiment, description is provided supposing that the entire target region is the ROI.

In subsequent Step S252, a sum of absolute values of amplitude values Aⱼₖ of the intermediate combined acoustic line signal at measurement points Pⱼₖ in the ROI is calculated and set as an energy value Eᵢ. Here, since the entire target region is the ROI, a sum of absolute values |Aⱼₖ| of amplitude values Aⱼₖ for all measurement points Pⱼₖ in the target region is calculated as an energy value E₁.

Subsequently, an evaluation value Kᵢ is calculated (Steps S253 through S255). Here, description is provided supposing that an evaluation value K₁ equaling 1.0 is calculated (Step S255).

Description continues referring to FIG. 3 once again. In Step S26, the evaluator 155 judges whether the evaluation value Kᵢ satisfies the predetermined condition. Here, description is provided supposing that the predetermined condition is that the evaluation value Kᵢ is lower than 0.8. Since the evaluation value K₁, which equals 1.0, does not satisfy the predetermined condition, a judgment is made in Step S27 of whether the transmission count i is smaller than maximum iₘₐₓ, and then a subsequent transmission event is performed when the transmission count i is smaller than maximum iₘₐₓ (Step S28).

Subsequently, a second transmission event is performed, in which an ultrasound beam is transmitted by using −5° being the second transmission angle (Step S22) and an acoustic line signal is generated (Step S23).

Further, in Step S24, an intermediate combined acoustic line signal is generated by the combining of acoustic line signals. Since i equals two here, combining is performed using the acoustic line signal generated in Step S23 for i=1 and an acoustic line signal generated in the immediately-preceding Step S23. As such, the combiner 153 combines these two acoustic line signals. Specifically, the combiner 153 reads out the intermediate combined acoustic line signal stored in the acoustic line signal holder 154, and generates a new intermediate combined acoustic line signal by, for each measurement point Pⱼₖ, summing a value for the measurement point Pⱼₖ in the intermediate combined acoustic line signal and a value for the measurement point Pⱼₖ in the acoustic line signal generated in the immediately-preceding Step S23, and performing division by two. The combiner 153 stores the new intermediate combined acoustic line signal so generated to the acoustic line signal holder 154.

In subsequent Step S25, an evaluation value is calculated based on the intermediate combined acoustic line signal stored in the acoustic line signal holder 154. First, in Step S251, data for the predetermined ROI is extracted from the intermediate combined acoustic line signal. Subsequently, in Step S252, a sum of absolute values of amplitude values Aⱼₖ of the intermediate combined acoustic line signal at measurement points Pⱼₖ in the ROI is calculated and set as an energy value Eᵢ. Here, since the entire target region is the ROI, a sum of absolute values |Aⱼₖ| of amplitude values Aⱼₖ for all measurement points Pⱼₖ in the target region is calculated as an energy value E₂.

Subsequently, an evaluation value Kᵢ is calculated (Steps S253 through S255). Here, the evaluation value Kᵢ is calculated by using the following expression.

Kᵢ=(Eᵢ−Eᵢ₋₁)/E₁

That is, the evaluation value Kᵢ is a value acquired by normalizing, by using the energy level of the acoustic line signal for transmission angle 0°, the contribution of Step S24 to the intermediate combined acoustic line signal stored in the acoustic line signal holder 154 in terms of energy.

Subsequently, in Step S26, the evaluator 155 judges whether the evaluation value Kᵢ satisfies the predetermined condition. Here, the predetermined condition is that the evaluation value Kᵢ is lower than 0.8. When evaluation value K₂ does not satisfy the predetermined condition, a judgment is made in Step S27 of whether the transmission count i is smaller than maximum iₘₐₓ, and then a subsequent transmission event is performed when the transmission count i is smaller than maximum iₘₐₓ (Step S28).

Subsequently, a p^{th} transmission event (where p is an integer that is 3 or greater) is similarly performed, in which an ultrasound beam is transmitted by using a p^{th} transmission angle (Step S22) and an acoustic line signal is generated (Step S23). Further, since i equals p, an intermediate combined acoustic line signal is generated in Step S24 by combining acoustic line signals generated in Step S23 for different values of i from one to p. Specifically, the combiner 153 generates a new intermediate combined acoustic line signal by reading the intermediate combined acoustic line signal stored in the acoustic line signal holder 154, and for each measurement point Pⱼₖ, (i) multiplying a value for the measurement point Pⱼₖ in the read intermediate combined acoustic line signal by (p−1) to acquire a sum of values for the measurement point Pⱼₖ for values of i from one to (p−1), (ii) adding the value for the measurement point Pⱼₖ in the acoustic line signal generated in the immediately-preceding Step S23, and (iii) performing division by p. The combiner 153 stores the new intermediate combined acoustic line signal so generated to the acoustic line signal holder 154. In Step S25, an evaluation value is calculated based on the intermediate combined acoustic line signal stored in the acoustic line signal holder 154. First, an energy value Eₚ is calculated through Steps S251 through S252. Subsequently, an evaluation value Kₙ is acquired by using Kₚ=(Eₚ−Eₚ₋₁)/E₁.

When an evaluation value K_{q} for a CO transmission event (where q is an integer that is 2 or greater) satisfies the predetermined condition, or that is, when the evaluation value K_{q} is lower than 0.8, the outputter 156 acquires the intermediate combined acoustic line signal stored in the acoustic line signal holder 154 and outputs the intermediate combined acoustic line signal as a combined acoustic line signal. Further, the outputter 156 sets zero to the combination count C stored in the acoustic line signal holder 154.

Finally, in Step S40, a B-mode image is generated from the combined acoustic line signal, and the B-mode image is output to the display 103. Specifically, the image processor 160 performs conversion of coordinates and conversion into luminance signals on the combined acoustic line signal to generate a B-mode image, and the display controller 170 causes the display 103 to display the B-mode image.

Note that when No in Step S27, or that is, when the transmission count i reaches maximum iₘₐₓ, processing similar to that performed when an evaluation value satisfies the predetermined condition (Yes in Step S26) is performed. This configuration is made to suppress excess repetition of transmission events. Note that maximum iₘₐₓ may for example be equal to the number of transmission angles included in the transmission angle sequence. When making this configuration, processing proceeds to a subsequent frame once transmission events have been performed by using all transmission angles included in the transmission angle sequence.

When proceeding to a subsequent frame, processing for the subsequent frame may be commenced in accordance with a predetermined frame rate. Alternatively, processing for the subsequent frame may be commenced as soon as Step S40 is completed or after a predetermined time amount elapses from when Step S40 is completed. The latter configuration achieves active improvement of frame rate particularly when the number of acoustic line signals combined is small, and thus achieves an improvement in temporal resolution.

### <Relation Between Transmission Count i and Evaluation Value Kᵢ>

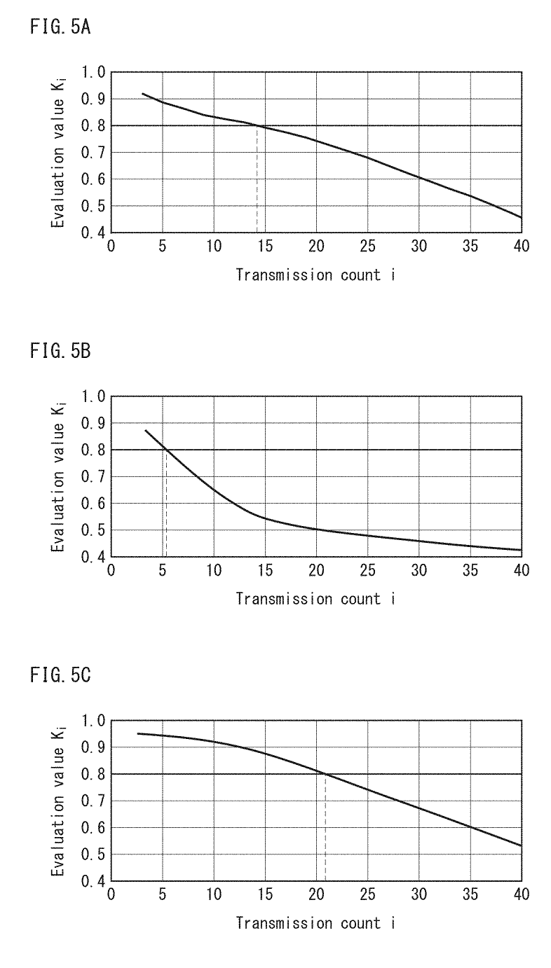

The following describes a relation between transmission count i and evaluation value Kᵢ, with reference to the relation graphs in FIGS. 5A through 5C.

FIG. 5A illustrates a general relation between transmission count i and evaluation value Kᵢ. The following explains why such a relation exists.

As described above, an evaluation value Kᵢ indicates the contribution of an i^{th} acoustic line signal to an i^{th} intermediate combined acoustic line signal in terms of energy, relative to energy value E₁ of an acoustic line signal acquired through a transmission event performed by using transmission angle 0°. Energy value E of an intermediate combined acoustic line signal is a sum of absolute values of amplitude values at measurement points Pⱼₖ. Thus, when two intermediate combined acoustic line signals for different values of i are similar to one another, energy values Eᵢ of the two intermediate combined acoustic line signals are also naturally similar. Accordingly, the greater the difference between an i^{th} acoustic line signal and an (i−1)^{th} intermediate combined acoustic line signal generated by combining acoustic line signals for the first through (i−1)^{th} transmission events, the greater the evaluation value Kᵢ.

Further, in a case where the difference between two successive acoustic line signals remains substantially the same from the first through i^{th} acoustic line signals, the difference between the (i−1)^{th} intermediate combined acoustic line signal and the i^{th} acoustic line signal decreases and approaches a certain value as the transmission count i increases. Accordingly, the evaluation value Kᵢ also decreases and approaches a certain value as the transmission count i increases. Accordingly, in such a case, the greater the transmission count i, the smaller the evaluation value Kᵢ. Further, the decrease in evaluation value Kᵢ brought about by the increase in transmission count i is monotonical, as illustrated in FIG. 5A.

Meanwhile, when there is a fast cyclic movement at a part of the target region, the relation is such as that illustrated in FIG. 5B. Specifically, when there is a fast movement at a part of the target region, the combining of acoustic line signals improves spatial resolution at parts of the target region where there is no movement, whereas the combining does not improve spatial resolution and yields an average of different states at the part of the target region where the movement is present. Further, when the movement is cyclic, almost no change in intermediate combined acoustic line signal energy occurs once combining is completed for half the cycle of the movement. Further, following this point, the change in intermediate combined acoustic line signal energy brought about by increasing the number of acoustic line signals combined is small, and the evaluation value Kᵢ decreases rapidly as the transmission count i increases. Due to this, the number of transmission events having been performed at the point when the evaluation value Kᵢ satisfies the predetermined condition is smaller in the case illustrated in FIG. 5B than in the case illustrated in FIG. 5A, provided that the same threshold is used. Accordingly, when a fast cyclic movement is present at a part of the target region, the number of transmission events performed for generating one frame decreases, and thus, a decrease in spatial resolution can be suppressed.

Further, when there is anisotropy in the target region, the relation is such as that illustrated in FIG. 5C. For example, suppose a case where the element line direction is the left-right direction, the travel direction of an ultrasound beam with transmission angle 0° is the bottom direction, and a bone exists in the right side of the target region in the left-right direction, reaching the right end of the target region. In this case, since the bone reflects ultrasound beams, a region below the bone is shaded by the bone. Accordingly, when using an ultrasound beam with transmission angle 0° or ultrasound beams directed to the bottom-left, not enough ultrasound reflection can be acquired from the region below the bone, and thus, spatial resolution would be low at that region. Meanwhile, when using ultrasound beams directed to the bottom-right, the region shaded by the bone changes in accordance with the ultrasound beam travel direction. Specifically, a part of a region that is shaded by the bone when the transmission angle is 0° would no longer be shaded by the bone when the transmission angle is 5°, and another part of the region that is shaded by the bone when the transmission angle is 0° would no longer be shaded when the transmission angle is 10°. Due to this, each time the combining is performed, intermediate combined acoustic line signal spatial resolution improves due to the shaded region being supplemented, and intermediate combined acoustic line signal energy value increases. As a result, until the supplementing is completed, a great decrease in evaluation value Kᵢ does not occur and evaluation value Kᵢ decreases gently as the transmission count i increases. Due to this, the number of transmission events having been performed at the point when the evaluation value Kᵢ satisfies the predetermined condition is greater in the case illustrated in FIG. 5C than in the case illustrated in FIG. 5A, provided that the same threshold is used. Accordingly, even when there is anisotropy in the target region, a sufficient level of spatial resolution can be ensured due to an increased number of transmission events being performed for generating one frame.

### <Order of Transmission Angles in Transmission Angle Sequence>

The following describes an order in which the transmission angles in the transmission angle sequence are arranged, with reference to FIG. 6.

Specifically, FIG. 6 is a schematic illustrating transmission angles for transmission events, regions for which acoustic line signals are generated, and regions covered by intermediate combined acoustic line signals. The following description supposes that the direction in which the five transducers 101a in the schematic form a line is the element line direction (x-axis direction) and that the normal direction with respect to the element line direction is the depth direction (y-axis direction).

The first transmission event is performed by using 0° as the transmission angle. As such, the ultrasound beam travel direction is parallel to the y axis. Here, description is provided supposing that all of the transducers 101a are driven at the same time so that spherical waves ci1 through ci5 from the transducers 101a, which form an ultrasound beam wavefront, have the same size. Accordingly, an acoustic line signal is generated based on ultrasound reflection from the entire target region.

The second transmission event is performed by using −5° as the transmission angle. Thus, the ultrasound beam travel direction is inclined with respect to the y axis by 5° in the x-axis negative direction. In order to achieve this, a tangential line of the spherical waves ci1 though ci5 from the transducers 101a needs to be inclined with respect to the x axis by 5° in the y-axis positive direction. Accordingly, delaying of drive timings of the transducers 101a is performed so that the transducers 101a are driven in order starting from the transducer 101a farthest in the x-axis positive direction to the transducer 101a farthest in the x-axis negative direction so that the radiuses of the semispherical waves decrease in the order of ci5, ci4, . . . , ci1. Accordingly, an acoustic line signal is generated based on ultrasound reflection from the entire target region, but a blank region (blank1) where no in-phase ultrasound beam passes through is produced at the bottom right side of the drawing, and spatial resolution is low at the blank region.

Similarly, in the third, fourth, and fifth transmission events, blank regions blank2, blank3, and blank4, where spatial resolution is low, are respectively produced.

In the present embodiment, the order in which transmission angles in the transmission angle sequence are arranged satisfy the following conditions: (i) the transmission angles alternate between positive and negative angles; (ii) the transmission angles are arranged so that the smaller the transmission angle, the earlier the position in the order; (iii) the first transmission angle is 0°. This is due to the following reasons.

(i) The transmission angles are arranged to alternate between positive and negative angles in order to prevent spatial biasing of blank regions to one side, namely the right side (x-axis positive side) or the left side (x-axis negative side). When there is an unbalance between the number of transmission events for positive transmission angles and the number of transmission events for negative transmission angle among the transmission events that have been performed at the point when the evaluation value Kᵢ satisfies the predetermined condition, there is a risk of spatial resolution not being uniform in the x-axis direction. Since spatial resolution at a blank region cannot be increased through the combining, the smaller the number of acoustic line signals for which a measurement point Pⱼₖ is not located in blank regions, the higher the spatial resolution of the combined acoustic line signal at the measurement point Pⱼₖ. Due to this, when blank regions in the acoustic line signals that are used in the combining are spatially biased, the combined acoustic line signal would have lower spatial resolution at a region corresponding to many blank regions than at other regions. As such, it is preferable to transmit ultrasound beams at a good balance in the left and right directions in order to avoid such spatial biasing of blank regions. Accordingly, ununiformity of combined acoustic line signal spatial resolution can be prevented, regardless of the number of transmission events having been performed at the point when the evaluation value Kᵢ satisfies the predetermined condition, by arranging transmission angles to alternate between positive angle and negative angles.

(ii) The transmission angles are arranged so that the smaller the transmission angle, the earlier the position in the order. This configuration is made in order to ensure that blank region area increases as transmission progresses rather than decreasing, and to thereby suppress an increase in the number of evaluation values Kᵢ with large values. Specifically, the greater the absolute value of a transmission angle, the greater the blank region area. Due to this, when performing transmission events using transmission angles in the order from those with large absolute values to those with small absolute values, a transmission event performed later produces acoustic line signal data for a part of a blank region for a transmission event performed earlier. This results in improvement of spatial resolution at the area. As a result, the evaluation value Kᵢ remains high due to the change in intermediate combined acoustic line signal energy value continuing to be great. That is, it takes a relatively long amount of time for improvement of spatial resolution brought about by the combining of acoustic line signals to reach saturation, and thus, the number of transmission events that need to be performed increases. Meanwhile, when arranging transmission angles so that the smaller the transmission angle, the earlier the position in the order, such supplementation of blank region is not likely to occur. Thus, improvement of spatial resolution brought about by the combining of acoustic line signals reaches saturation easily, and it becomes easier to suppress the number of transmission events that are performed.

Further, when controlling transmission angles according to the conditions (i) and (ii) described above, it is preferable to configure the transmission angle sequence so that the first transmission angle is 0°.

### <Summary>

The ultrasound signal processing device and the ultrasound diagnostic device pertaining to embodiment 1 reduce the number of transmission events while maintaining spatial resolution of a certain level or higher. Further, the ultrasound signal processing device and the ultrasound diagnostic device achieve improved temporal resolution when configured to actively change frame rate in accordance with the number of transmission events.

### <Modification 1>

The ultrasound signal processing device and the ultrasound diagnostic device pertaining to embodiment 1 calculate an energy value E for an intermediate combined acoustic line signal based on a sum of amplitude values of the intermediate combined acoustic line signal, and calculate an evaluation value for the intermediate combined acoustic line signal Kᵢ from a degree of change of the energy value Eᵢ.

Meanwhile, in modification 1, an energy value E for an intermediate combined acoustic line signal is calculated based on a spectrum of the intermediate combined acoustic line signal, and the evaluation value Kᵢ for the intermediate combined acoustic line signal is calculated from a degree of change of the energy value Eᵢ.

### <Operations>

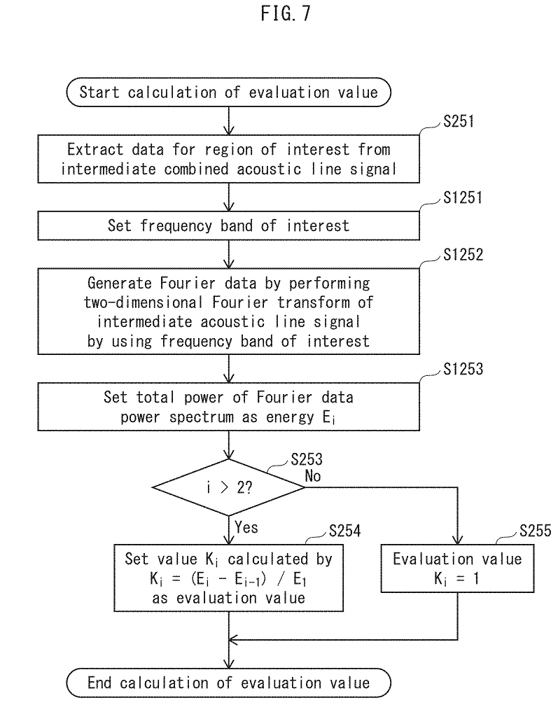

FIG. 7 is a flowchart illustrating an evaluation value calculation operation pertaining to modification 1. Note that operations already illustrated in FIG. 4 are indicated by using the same step numbers and description thereof is not provided in the following.

First, in Step S251, data pertaining to the predetermined ROI is extracted from the intermediate combined acoustic line signal. In the present modification, description is provided supposing that the entire target region is the ROI.

In subsequent Step S1251, a frequency band of interest is set. The frequency band of interest specifies frequency components to be used for the calculation of energy value, among frequency components of the intermediate combined acoustic line signal. In the following, description is provided supposing that the frequency band from 4 MHz to 7 MHz is set as the frequency band of interest, considering frequencies of transmitted ultrasound beams. The use of such a frequency band prevents the evaluation of the intermediate combined acoustic line signal from being influenced by low frequency and high frequency noises. However, the frequency band of interest need not be set to the above-described example. Alternatively, a frequency band differing from that described above may be set as the frequency band of interest, or the frequency band of interest may be set to cover all frequencies.

In subsequent Step S1252, two-dimensional Fourier transform is performed on the intermediate combined acoustic line signal based on the frequency band of interest, to generate Fourier data.

In subsequent Step S1253, total power of a power spectrum of the Fourier data is calculated as the energy value Eᵢ. Here, since the frequency band of interest is 4 MHz to 7 MHz, the energy value E is a value acquired by calculating the total power of the power spectrum within the frequency range of 4 MHz to 7 MHz.

Subsequently, the evaluation value Kᵢ for the intermediate combined acoustic line signal is calculated (Steps S253 through S255). Specifically, the evaluation value Kᵢ is a value acquired by normalizing, by using the energy value calculated in Step S1253 for i=1 (i.e., for transmission angle 0°), the contribution of Step S24 to the intermediate combined acoustic line signal in terms of power within the frequency range of 4 MHz to 7 MHz.

### <Summary>

The above-described structure allows eliminating the influence of changes brought about by low frequency and high frequency noises in an intermediate combined acoustic line signal in the calculation of the evaluation value Kᵢ for the intermediate combined acoustic line signal. Accordingly, the structure allows detecting with high accuracy whether intermediate combined acoustic line signal spatial resolution improves through the combining of acoustic line signals.

### <Modification 2>

The ultrasound signal processing device and the ultrasound diagnostic device pertaining to modification 1 calculate an energy value E for an intermediate combined acoustic line signal based on a spectrum of the intermediate combined acoustic line signal, and calculates an evaluation value Kᵢ for the intermediate combined acoustic line signal from a degree of change of the energy value Eᵢ.

Meanwhile, in modification 2, one-dimensionalizing of an intermediate combined acoustic line signal is performed, an energy value E for the intermediate combined acoustic line signal is calculated based on a spectrum of the one-dimensional intermediate combined acoustic line signal, and an evaluation value Kᵢ for the intermediate combined acoustic line signal is calculated from a degree of change of the energy value Eᵢ.

### <Operations>

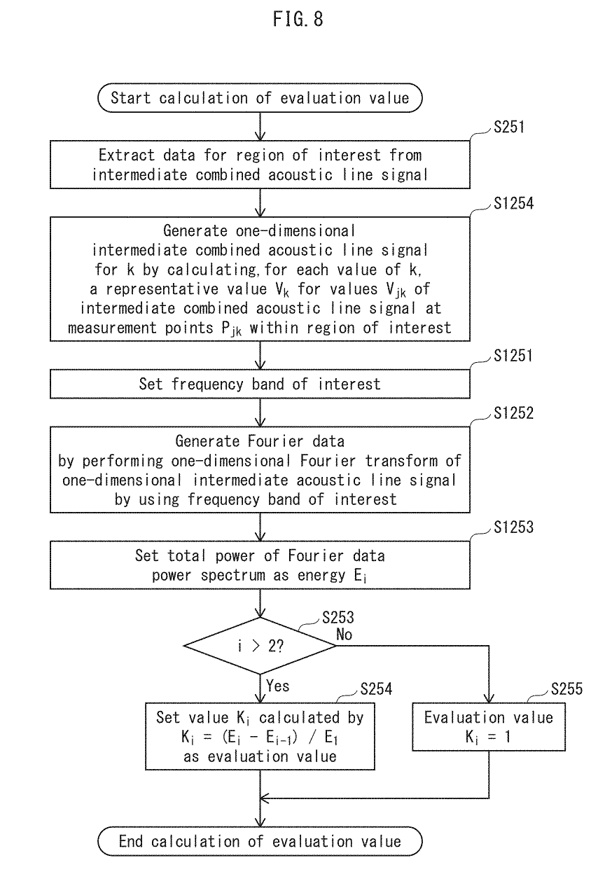

FIG. 8 is a flowchart illustrating an evaluation value calculation operation pertaining to modification 2. Note that operations already illustrated in FIGS. 4 and 7 are indicated by using the same step numbers and description thereof is not provided in the following.

First, in Step S251, data pertaining to the predetermined ROI is extracted from the intermediate combined acoustic line signal. In the present modification, description is provided supposing that the entire target region is the ROI.

In subsequent Step S1254, the intermediate combined acoustic line signal is one-dimensionalized. Specifically, the following processing is performed. First, an evaluator calculates, for each depth-direction coordinate k, a representative value Vₖ of values Vⱼₖ of the intermediate combined acoustic line signal at measurement points Pⱼₖ in the ROI with the depth-direction coordinate k. Specifically, for measurement points Pⱼₗ whose depth-direction coordinate k is one, the evaluator sets a maximum of the values Vⱼₗ of the intermediate combined acoustic line signal as V₁. The evaluator performs this processing for other values of k (i.e., k=2, k=3, . . . ,) and thereby calculates value Vₖ for each k. Finally, by indicating Vₖ as a function of k, the evaluator generates a one-dimensional intermediate combined acoustic line signal.

In subsequent Step S1251, a frequency band of interest is set. The frequency band of interest specifies frequency components to be used for the calculation of energy level, among frequency components of the intermediate combined acoustic line signal. In the following, description is provided supposing that the frequency band from 4 MHz to 7 MHz is set as the frequency band of interest, considering frequencies of transmitted ultrasound beams.

In subsequent Step S1255, one-dimensional Fourier transform is performed on the intermediate combined acoustic line signal based on the frequency band of interest to generate Fourier data.

In subsequent Step S1253, total power of a power spectrum of the Fourier data is calculated as the energy value Eᵢ. Here, since the frequency band of interest is 4 MHz to 7 MHz, the energy value Eᵢ is a value acquired by calculating the total power of the spectrum within the frequency range of 4 MHz to 7 MHz.

Subsequently, the evaluation value Kᵢ is calculated (Steps S253 through S255). Specifically, the evaluation value Kᵢ is a value acquired by normalizing, by using the energy value calculated in Step S1253 for i=1 (i.e., for transmission angle) 0°, the contribution of Step S24 to the one-dimensional intermediate combined acoustic line signal in terms of power within the frequency range of 4 MHz to 7 MHz.

### <Summary>

The above-described structure allows calculating an evaluation value Kᵢ for an intermediate combined acoustic line signal based on a change in maximums of values of the intermediate combined acoustic line signal in the frequency band of interest. Accordingly, the structure prevents the evaluation value Kᵢ from taking a large value in a case, such as when there is a movement or the like, where values of the intermediate combined acoustic line signal change but the maximums of the values do not change, in which case spatial resolution would not improve by the combining of acoustic line signals. Accordingly, the structure allows detecting with high accuracy whether intermediate combined acoustic line signal spatial resolution improves through the combining of acoustic line signals.

### Embodiment 2

In embodiment 1, description is provided of a case where the number of transmission events is changed actively frame by frame.

Meanwhile, embodiment 2 is characterized in that setting of the number of transmission events is performed every few frames.

### <Structure>

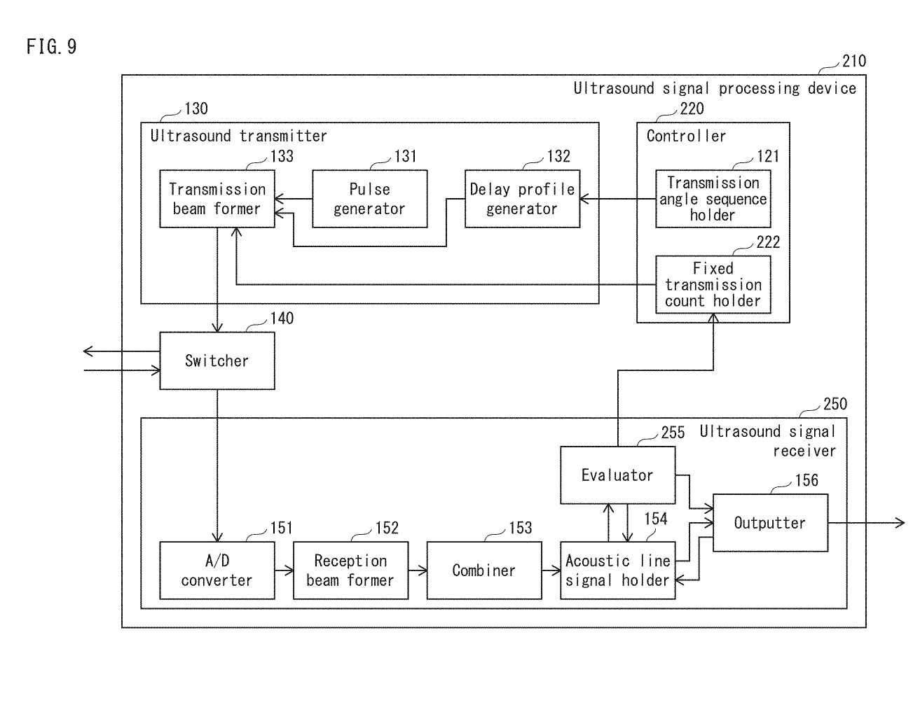

FIG. 9 is a functional block diagram illustrating an ultrasound signal processing device 210 pertaining to embodiment 2. Note that constituent elements already illustrated in FIG. 2 are indicated by using the same reference signs and description thereof is not provided in the following.

The ultrasound signal processing device 210 includes a controller 220 in place of the controller 120 and an ultrasound receiver 250 in place of the ultrasound receiver 150. The ultrasound receiver 250 includes an evaluator 255. The evaluator 255 stores the transmission count i at the point when an evaluation value of an intermediate combined acoustic line signal satisfies the predetermined condition to a transmission count holder 222 of the controller 220 as a fixed transmission count.

The controller 220 has the following functions in addition to the above-described functions of the controller 120. Upon commencing processing for a frame, the controller 220 judges whether the current frame is a frame at which a new fixed transmission count is to be set. When judging that the current frame is not a frame at which a new fixed transmission count is to be set, the controller 220 causes the ultrasound transmitter 130 to execute the number of transmission events indicated by a fixed transmission count currently stored in the transmission count holder 222. Meanwhile, when judging that the current frame is a frame at which a new fixed transmission count is to be set, the controller 220 causes the transmission count holder 222 to store a new fixed transmission count.

The evaluator 255 has the following functions in addition to the above-described functions of the evaluator 155. In processing a frame at which a new fixed transmission count is to be set, the evaluator 255 outputs the transmission count i at which the evaluation value Kᵢ satisfies the predetermined condition as a new fixed transmission count to the transmission count holder 222. Meanwhile, in processing a frame at which a new fixed transmission count is not to be set, the evaluator 255 does not perform the calculation of the energy value Eᵢ or the evaluation value Kᵢ, and instead, causes the combiner 153 to generate an intermediate combined acoustic line signal by combining a number of acoustic line signals corresponding to the fixed transmission count currently stored by the transmission count holder 222 and causes the outputter 156 to unconditionally output the intermediate combined acoustic line signal as the combined acoustic line signal.

### <Operations>

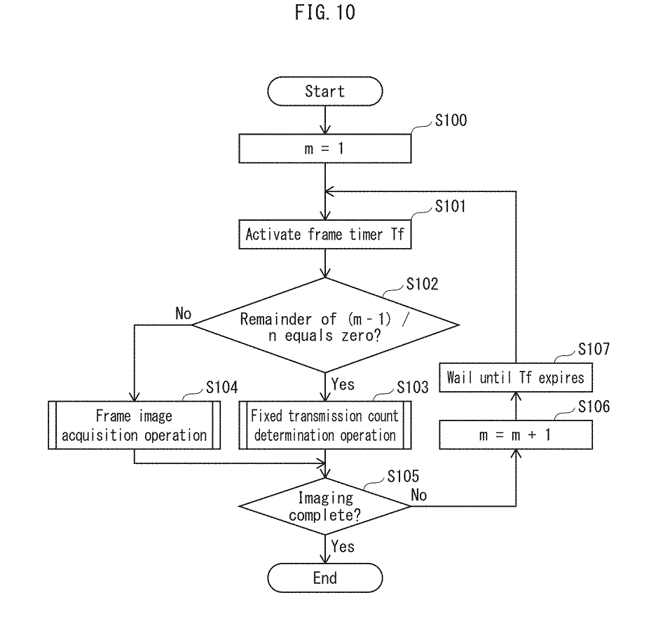

The following describes operations of an ultrasound diagnostic device pertaining to embodiment 2. FIG. 10 is a flowchart illustrating operations for multiple frames, performed by the ultrasound diagnostic device.

First, in Step S100, a frame counter value m is initialized. In subsequent Step S101, a frame timer Tf is activated.

In subsequent Step S102, a judgment is made of whether the frame counter value m satisfies a condition that a remainder of (m−1)/n equals zero. Here, n is a value indicating how often the operation for setting a new fixed transmission count (referred to in the following as a fixed transmission count determination operation) is to be performed in terms of number of frames, and for example is ten. When m equals one, the above-described condition is satisfied. Thus, the operation in Step S103 is subsequently performed.

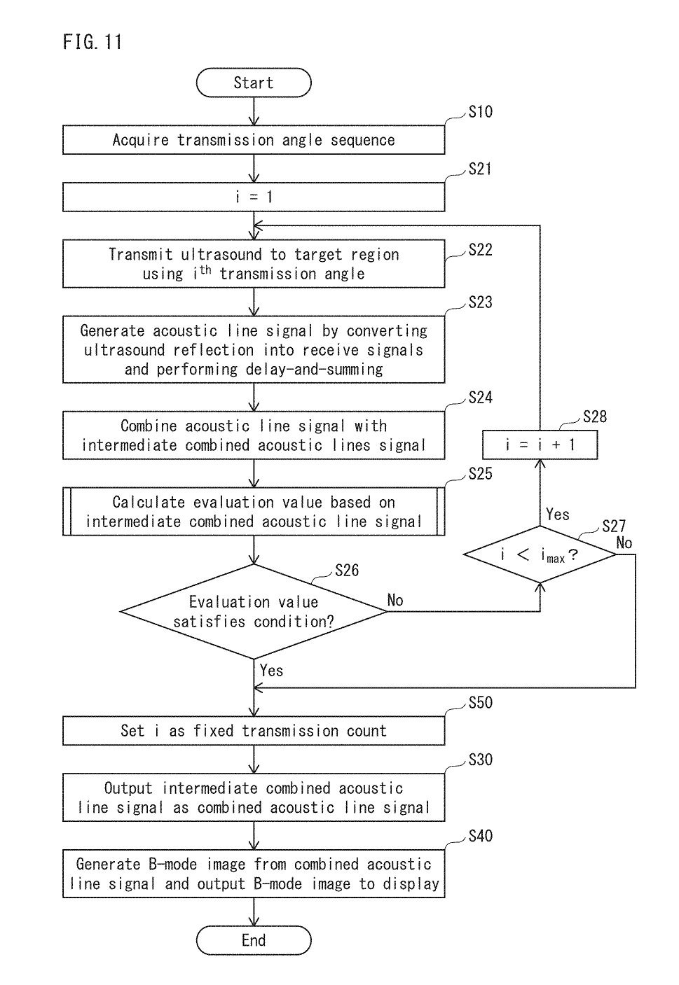

In Step S103, the ultrasound diagnostic device performs the fixed transmission count determination operation. FIG. 11 is a flowchart illustrating operations of the ultrasound diagnostic device for a frame for which the fixed transmission count determination operation is performed. Note that operations already illustrated in FIG. 4 are indicated by using the same step numbers and description thereof is not provided in the following.

The operations in the fixed transmission count determination operation in Step S103 are substantially similar to the operations of the ultrasound diagnostic device 100 pertaining to embodiment 1. However, in Step S50, the evaluator 255 stores value i when the evaluation value Kᵢ satisfies the predetermined condition as a new fixed transmission count to the transmission count holder 222. Accordingly, transmission count i at Step S103 is stored to the transmission count holder 222 as a new fixed transmission count.

Description continues referring to FIG. 10 once again. When there is a subsequent frame (No in Step S105), the frame counter value m is incremented (Step S106), and the ultrasound diagnostic device waits until expiration of the timer Tf (Step S107). Here, the timer Tf expires when an inter-frame interval in accordance with frame rate elapses. In subsequent Step S101, the frame timer Tf is activated once again.

In subsequent Step S102, a judgment is made of whether the frame counter value m satisfies the condition that the remainder of (m−1)/n equals zero. Since n equals ten, the condition is not satisfied when m equals two. Thus, in this case, the operation of Step S104 is subsequently performed.

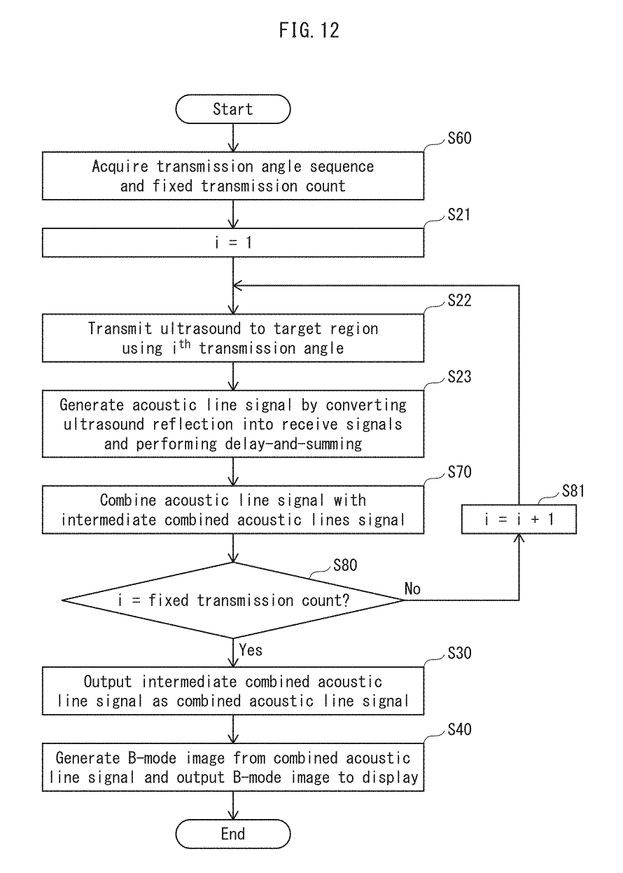

In Step S104, the ultrasound diagnostic device performs an image acquisition operation based on the fixed transmission count currently stored in the transmission count holder 222. FIG. 12 is a flowchart illustrating operations of the ultrasound diagnostic device for a frame for which the image acquisition operation is performed. Note that operations already illustrated in FIGS. 4 and 11 are indicated by using the same step numbers and description thereof is not provided in the following.

First, the ultrasound transmitter 130 acquires the transmission angle sequence from the transmission angle sequence holder 121 and acquires the fixed transmission count currently stored in the transmission count holder 222 in Step S60. Description is provided in the following supposing that the acquired transmission angle sequence is {0, −5, +5, −10, +10, −15, +15, . . . , −40, +40}, and supposing that the fixed transmission count currently stored in the transmission count holder 222 is seven.

Subsequently, one is set to the counter that counts the transmission count i (i.e., the counter is initialized) (Step S21), and an ultrasound beam is transmitted to the target area by using the i^{th} transmission angle (Step S22). Here, since i equals one, an ultrasound beam is transmitted by using the first transmission angle, namely 0°, as the transmission angle.

In subsequent Step S23, ultrasound reflection is converted into receive signals, and an acoustic line signal is generated through delay-and-summing.

In subsequent Step S70, an intermediate combined acoustic line signal is generated through the combining of acoustic line signals. The combiner 153 combines all acoustic line signals corresponding to values of i from one to the current value. Since i equals one here, the combining is performed using only the acoustic line signal having been generated in the immediately-preceding Step S23. Thus, the combiner 153 stores the acoustic line signal as-is to the acoustic line signal holder 154 as an intermediate combined acoustic line signal.

In subsequent Step S80, a judgment is made of whether i has reached the fixed transmission count acquired in Step S60. Here, since i has not yet reached the fixed transmission count, i is incremented in Step S81 and a subsequent transmission event is performed.

Following this, transmission events are repeatedly performed by incrementing i until i reaches the fixed transmission count (seven in this example).

In Step S80 after the 7^{th} transmission event, i reaches the fixed transmission count. Accordingly, processing proceeds to Step S30, where the outputter 156 acquires the intermediate combined acoustic line signal from the acoustic line signal holder 154 and outputs the intermediate combined acoustic line signal as a combined acoustic line signal. Here, the outputter 156 resets the combination count C stored in the acoustic line signal holder 154 to zero.

Finally, in Step S40, a B-mode image is generated from the combined acoustic line signal, and the B-mode image is output to the display 103. Specifically, the image processor 160 performs conversion of coordinates and conversion into luminance signals on the combined acoustic line signal to generate a B-mode image, and the display controller 170 causes the display 103 to display the B-mode image.

Through such operations, a combined acoustic line signal is generated in accordance with the fixed transmission count currently stored in the transmission count holder 222 and the transmission angle sequence.

Description continues referring to FIG. 10 once again. When there is a subsequent frame (No in Step S105), the frame counter value m is incremented (Step S106), and the ultrasound diagnostic device waits until expiration of the timer Tf (Step S107). Here, the timer Tf expires when an inter-frame interval in accordance with frame rate elapses. In subsequent Step S101, the frame timer Tf is activated once again. This results in operations for a third frame being commenced.

Through such processing, the fixed transmission count determination operation is performed once every n frames, and the image acquisition operation is performed for frames other than those for which the fixed transmission count determination operation is performed.

### <Summary>

The ultrasound signal processing device and the ultrasound diagnostic device pertaining to embodiment 2 perform the fixed transmission count determination operation for only some frames, and thus, the calculation of evaluation value does not need to be performed for every transmission event. Thus, the ultrasound signal processing device and the ultrasound diagnostic device achieve a reduction in computation amount. Further, the ultrasound signal processing device and the ultrasound diagnostic device prevent a frame-by-frame change in the number of transmission events that may occur when for example swinging of the ultrasound probe takes place, and thus, suppress violent fluctuations in computation amount and frame rate.

### <Modification 3>

In embodiment 2, description is provided of a case where frame rate remains fixed, regardless of a change in fixed transmission count. Meanwhile, the present modification describes a case where frame rate is changed in accordance with the fixed transmission count.

### <Operations>

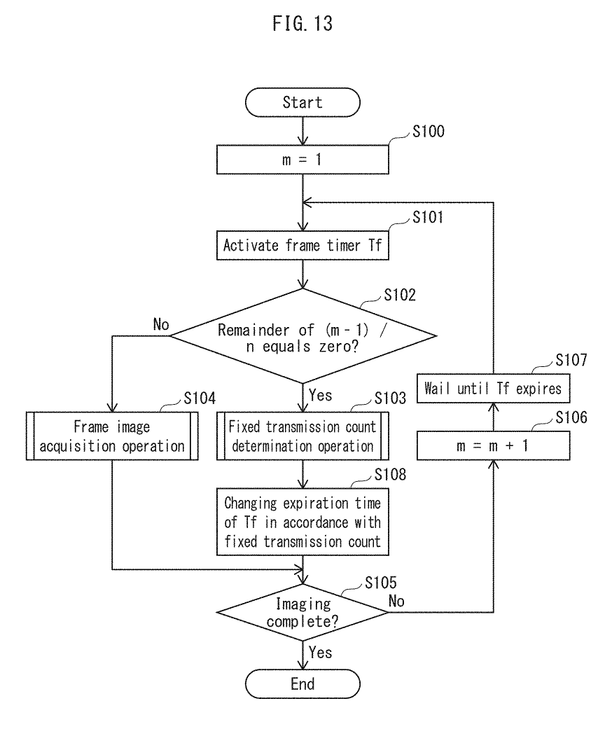

The following describes operations of an ultrasound diagnostic device pertaining to modification 3. FIG. 13 is a flowchart illustrating operations for multiple frames, performed by the ultrasound diagnostic device. Note that operations already illustrated in FIG. 10 are indicated by using the same step numbers and description thereof is not provided in the following.

First, in Step S100, a frame counter value m is initialized. In subsequent, Step S101, a frame timer Tf is activated.

In subsequent Step S102, a judgment is made of whether the frame counter value m satisfies the condition that the remainder of (m−1)/n equals zero. Here, n is a value indicating how often the fixed transmission count determination operation is to be performed in terms of number of frames, and for example is ten. When m equals one, the above-described condition is satisfied. Thus, the operation in Step S103 is subsequently performed.

In Step S103, the ultrasound diagnostic device performs the fixed transmission count determination operation.

In subsequent Step S108, the expiration time of the frame timer Tf is changed in accordance with the fixed transmission count set in Step S103. For example, supposing that the initial expiration time of the frame timer Tf is 50 ms (20 fps) and that the frame rate can be increased to 40 fps according to the fixed transmission count set in Step S103, the expiration time of the frame timer Tf is changed to 25 ms. Note that this change is applied even to a timer having being activated in immediately-preceding Step S101 and that is currently operating in Step S108.

When there is a subsequent frame (No in Step S105), the frame counter value m is incremented (Step S106), and the ultrasound diagnostic device waits until expiration of the timer Tf (Step S107). Here, the timer Tf expires when an inter-frame interval in accordance with the frame rate elapses. In subsequent Step S101, the frame timer Tf is activated.

In subsequent Step S102, a judgment is made of whether the frame counter value m satisfies the condition that the remainder of (m−1)/n equals zero. Since n equals ten here, the above-described condition is not satisfied when m equals two. Thus, in this case, the operation of Step S104 is subsequently performed.

In Step S104, the ultrasound diagnostic device performs the image acquisition operation based on the fixed transmission count currently stored in the transmission count holer 222.

When there is a subsequent frame (No in Step S105), the frame counter value m is incremented (Step S106), and the ultrasound diagnostic device waits until expiration of the timer Tf (Step S107). Here, the timer Tf expires when the inter-frame interval in accordance with the frame rate elapses. In subsequent Step S101, the frame timer Tf is activated. This results in operations for a third frame being commenced.

Through such processing, the fixed transmission count determination operation is performed once every n frames, and also, a frame rate to be applied starting at the current frame and until commencement of the subsequent fixed transmission count determination operation is determined. Accordingly, temporal resolution can be improved particularly when a small fixed transmission count is set.

### <Supplementary Explanation of Frame Rate>

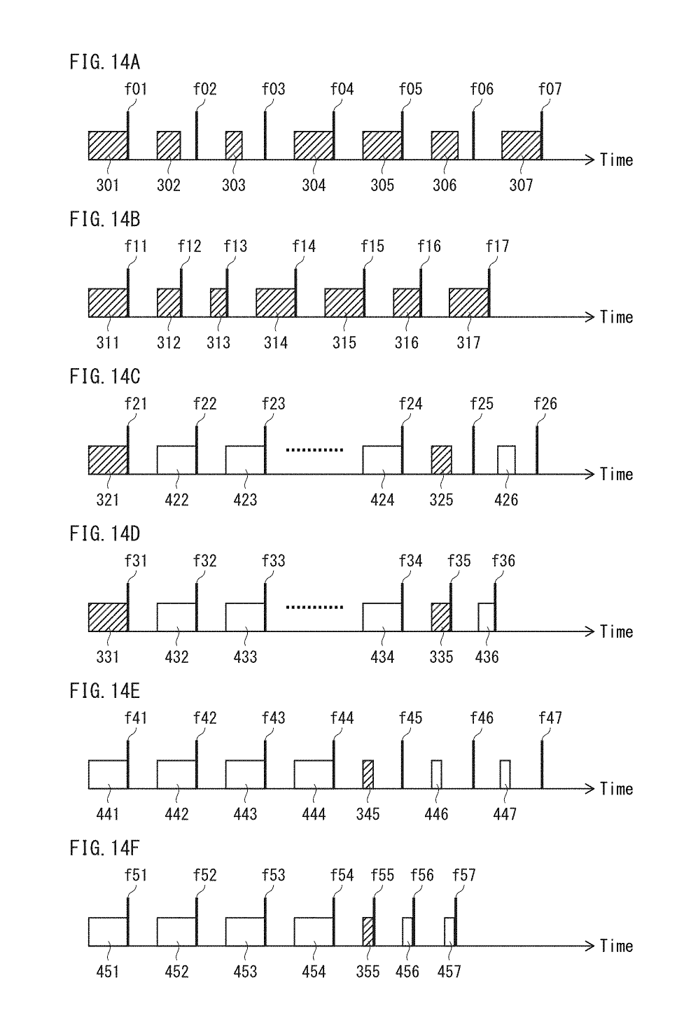

The following describes the relation between frame rate and the fixed transmission count determination operation, with reference to the timing charts of FIGS. 14A through 14F. Each of FIGS. 14A through 14F is a timing chart illustrating timings of fixed transmission count determination operations and image acquisition operations, and image output timings.

The ultrasound diagnostic device pertaining to embodiment 1 operates as illustrated in FIG. 14A or FIG. 14B. Specifically, when not actively changing frame rate, the interval between image output timings f01 through f07 is fixed, and frame operations 301 through 307 are each performed when a predetermined time amount elapses from an image output timing, as illustrated in FIG. 14A. Meanwhile, when actively changing frame rate, frame operations 311 through 317 are each performed when a predetermined time amount elapses from a previous image output timing, and image output timings f11 through f17 each immediately follow a frame operation, as illustrated in FIG. 14B. In this case, the time amount between an image output timing for each frame operation and commencement of a subsequent frame operation is fixed, and thus frame rate is dependent upon time amount required for the frame operation.

Meanwhile, the ultrasound diagnostic device pertaining to embodiment 2 operates as illustrated in FIG. 14C. Specifically, the fixed transmission count determination operation is performed at a regular basis, with operations performed for example in the order of fixed transmission count determination operation 321, image acquisition operations 422, 423, . . . , 424, fixed transmission count determination operation 325, and image acquisition operation 426. Further, the interval between image output timings f21 through f26 is fixed, and either a fixed transmission count determination operation or an image acquisition operation is performed when a predetermined time amount elapses from an image output timing.

Meanwhile, the ultrasound diagnostic device pertaining to modification 3 operates as illustrated in FIG. 14D. Specifically, the fixed transmission count determination operation is performed at a regular basis, with operations performed for example in the order of fixed transmission count determination operation 331, image acquisition operations 432, 433, . . . , 434, fixed transmission count determination operation 335, and image acquisition operation 436. Meanwhile, image output timings f31 through f37 each immediately follow a fixed transmission count determination operation or an image acquisition operation. In this case, frame rate remains the same from immediately after a fixed transmission count determination operation until immediately before a subsequent fixed transmission count determination operation, and frame rate may be changed each time a fixed transmission count determination operation is performed.

Note that while modification 3 describes that the fixed transmission count determination operation is performed every n frames, the fixed transmission count determination operation may alternatively be performed each time a predetermined time amount (for example, 0.2 seconds) elapses, or each time a user instruction is provided. For example, operations when performing the fixed transmission count determination operation each time a user instruction is provided and when not changing frame rate would be as illustrated in FIG. 14E. Specifically, in this case, the interval between image output timings f41 through f47 is fixed, and image acquisition operations 441 through 444, 446, and 447 are each performed when a predetermined time amount elapses from an image output timing. Meanwhile, when input received from a user indicates that the fixed transmission count determination operation is to be performed, a fixed transmission count determination operation 345 is performed immediately following image output timing f44, instead of the image acquisition operation. Meanwhile, when changing frame rate, frame rate is changed after commencement of fixed transmission count determination operation 355, as illustrated in FIG. 14F. Further, while not illustrated in FIGS. 14A through 14F, a modification may be made such that the fixed transmission count determination operation is performed every n frames and additionally performed whenever a user instruction is provided. A further modification can also be made such that when a fixed transmission count determination operation is to be performed based on a user instruction, the fixed transmission count determination operation is performed after n frames from the immediately-preceding fixed transmission count determination operation.

### Other Modifications Pertaining to Embodiments

(1) In the embodiments and the modifications, the combiner stores an intermediate combined acoustic line signal corresponding to values of i from one to p to the acoustic line signal holder. Further, in combining an acoustic line signal for p+1, the combiner multiples values in the intermediate combined acoustic line signal by p, adds corresponding values in the acoustic line signal for p+1, and performs division by using p+1. The combiner generates a new intermediate combined acoustic line signal in such a manner, and stores the new intermediate combined acoustic line signal to the acoustic line signal holder. However, the combining need not be performed as described above. For example, the combiner may store, to the acoustic line signal storage, a summed acoustic line signal acquired by simply summing corresponding values in the acoustic line signals for values of i from one to p, in which case the evaluator and the outputter may acquire an intermediate combined acoustic line signal by dividing values in the summed acoustic line signal by a combination count. Note that the acquisition of the intermediate combined acoustic line signal, in either of the cases described above, need not be performed by only simply summing corresponding values in acoustic line signals. For example, weighting based on transmission angles may be performed, and/or exclusion of blank regions regarding that there is no data for the blank regions may be performed.

Further, in embodiment 2 and modification 3, the combiner also generates an intermediate combined acoustic line signal in the image acquisition operation, by performing processing similar to that in the fixed transmission count determination operation. However, for example, a modification may be made such that in the image acquisition operation, the combiner holds a number of acoustic line signals corresponding to the fixed transmission count and combines these acoustic line signals.

(2) In the embodiments and modifications, {0, −5, +5, −10, +10, −15, +15, . . . , −40, +40} is used as an example of the transmission angle sequence. However, the transmission angle sequence may for example be {0, −0.2, +0.2, −0.4, +0.4, . . . } or {0, −0.3, +0.3, −0.6, +0.6, . . . }. Further, the transmission angles in the transmission angle sequence need not be arranged in the order of {0, negative angle, positive angle, negative angle, positive angle, . . . }, and may be arranged in an order such as {0, positive angle, negative angle, positive angle, negative angle, . . . }, {0, positive angle, negative angle, negative angle, positive angle, positive angle, negative angle, negative angle, . . . }, or {0, negative angle, positive angle, positive angle, negative angle, negative angle, positive angle, positive angle, . . . }. Further, the transmission angles in the transmission angle sequence need not be arranged in the order of increasing absolute value, and may be for example arranged in an order such as {0, +8, −8, +4, −4, +6, −6, +2, −2, . . . }, where the fourth and subsequent transmission angles are set to be within a range defined by the first through third transmission angles.

Further, when the transmission angle sequence holder stores multiple transmission angle sequences, the controller may determine which of the transmission angle sequences to use based on target region area, position of target region in subject, tissue inside target region, etc. Configuring the controller to perform such control allows, for example, using a different, suitable transmission angle sequence for the imaging of fingers for examination of rheumatism and the imaging of internal organs for examination of cancer.

(3) In embodiment 1, modifications 1 and 2, and the fixed transmission count determination operation in embodiment 2 and modification 3, the combiner generates an intermediate combined acoustic line signal and the evaluator calculates an evaluation value Kᵢ for the intermediate combined acoustic line signal and judges whether the evaluation value Kᵢ satisfies a predetermined condition, each time a transmission event is performed. However, the judgment using an evaluation value for an intermediate combined acoustic line signal need not be performed for every transmission event. For example, the judgment may be performed only when the number of transmission events having been performed satisfies a predetermined condition, such that the judgment is performed every two or four transmission events. When making such a modification, it suffices for the evaluator to perform the calculation of evaluation value only for transmission events for which the judgment is to be performed. Further, when performing the calculation of evaluation value and judgment using the evaluation value every r transmission events (where r is an integer that two or greater), the evaluation value Kᵢ may be defined by using the following expression: Kᵢ=(Eᵢ−Eᵢ₋ᵣ)/E₁. Further, the combiner, instead of performing the combining to generate an intermediate combined acoustic line signal for every transmission event, may perform the combining only for each transmission event for which the judgment is to be performed. By performing such processing, computation amount for calculating evaluation values can be reduced.

(4) In embodiment 1, modifications 1 and 2, and the fixed transmission count determination operation in embodiment 2 and modification 3, an evaluation value Kᵢ is calculated by using energy value E₁ of a first acoustic line signal as a reference, but the calculation may be performed by using an energy value of an acoustic line signal corresponding to transmission angle 0° as a reference. Further, note that when the first transmission angle in the transmission angle sequence is not 0°, an energy value E₁ of the first acoustic line signal may be simply used as a reference, or an energy value of a second or subsequent acoustic line signal corresponding to transmission angle 0° may be used as a reference. Further, when the transmission angle sequence does not include 0°, an energy value E₁ of the first acoustic line signal may be simply used as a reference, or an energy value of a second or subsequent acoustic line signal corresponding to a transmission angle with a smallest absolute value may be used as a reference. In any case, it is preferable that the energy value used as the reference have as large an energy value as possible, and thus, it is preferable that an energy value of an acoustic line signal corresponding to a transmission angle having as small an absolute value as possible be used.

(5) In embodiment 1, modifications 1 and 2, and the fixed transmission count determination operation in embodiment 2 and modification 3, the evaluator calculates an evaluation value Kᵢ for an intermediate combined acoustic line signal covering the entire target region. Alternatively, for example, the evaluator may calculate an evaluation value Kᵢ for an intermediate combined acoustic line signal by using data of the intermediate combined acoustic line signal corresponding to the ROI, which is a part of the target region. For example, the ROI may be a central sub-region when splitting the target region into 3×3 sub-regions. Alternatively, the ROI may be a region of a predetermined size with its center located at the center of the target region. By performing processing in such manner, computation amount for calculating evaluation values can be reduced.

(6) In each of the embodiments and modifications, the ultrasound diagnostic device transmits plane wave ultrasound beams. However, the ultrasound beams transmitted need not be plane waves, and may be unfocused ultrasound waves with any shape. For example, the transmitted ultrasound beams may be diffused ultrasound beams with convex wavefronts.

(7) In each of the embodiments and the modifications, the ultrasound diagnostic device displays B-mode images on the display. However, for example, the ultrasound diagnostic device may output B-mode image data to a storage and/or other devices. Alternatively, for example, the ultrasound diagnostic device may output combined acoustic line signals to a storage and/or other devices.

(8) In each of the embodiments and the modifications, the ultrasound diagnostic device is connectable to the display. However, the ultrasound diagnostic device does not need to be connectable to the display. For example, the ultrasound diagnostic device may include the display as a built-in component. Similarly, the ultrasound diagnostic device may include the ultrasound probe as a built-in component, or alternatively, the ultrasound diagnostic device may not include the A/D converter or the receive beam former and may acquire acoustic line signals from an ultrasound probe including the A/D converter and the receive beam former.

(9) All or some of the constituent elements of the ultrasound diagnostic devices pertaining to the embodiments and the modifications may be implemented as one or more chips of integrated circuits, may be implemented as a computer program, or may be implemented in any other form. For example, a modification may be made of implementing the evaluator and the outputter as a single chip, or a modification may be made of implementing the ultrasound transmitter on one chip and implementing the ultrasound receiver and other constituent elements on another chip.

Implementation with an integrated circuit is typically achieved by using a large scale integration (LSI). An LSI may be referred to as an integrated circuit, a system LSI, a super LSI, or an ultra LSI may be used depending on the level of integration.

Further, techniques of circuit integration are not limited to LSI, and implementation may be achieved by a dedicated circuit or general-purpose processor. Further, a field programmable gate array (FPGA) that is programmable after LSI manufacture or a reconfigurable processor, in which circuit cell connections and settings in the LSI can be reconfigured after LSI manufacture may be used.

Further, if a circuit integration technology is introduced that replaces LSI due to advances in semiconductor technology or another derivative technology, such technology may of course be used to integrate the functional blocks.

Further, the ultrasound diagnostic devices pertaining to the embodiments and modifications may be implemented as a program stored on a storage medium and a computer that reads and executes the program. The storage medium may be any kind of storage medium, such as a memory card or DVD-ROM. Further, the ultrasound diagnostic device pertaining to the present invention may be implemented as program downloadable via a network and a computer that downloads and executes the program.

(10) The above embodiments each describe a preferable and specific example of the present invention. The values, shapes, materials, constituent elements, positions and connections of the constituent elements, processes, ordering of processes, etc., are only examples and are not intended to limit the scope of the present invention. Further, among the constituent elements described in the embodiments, those not recited in the independent claims that indicate highest level concepts of the present invention are described as optional elements constituting a preferable form.

Further, in order to aid understanding of the invention, the dimensions of the constituent elements illustrated in the drawings referred to in the embodiments may differ from actual dimensions. Further, the present invention is not intended to be limited in scope by the description in the embodiments, and can be appropriately modified so as not to depart from the scope of the present invention.

Further, in ultrasound diagnostic devices are members such as circuit elements and lead lines on substrates, but description thereof is omitted, as various forms of implementation of electrical wiring and circuitry are possible based on common knowledge in the technical fields, and such description is not directly relevant to the present invention. The drawings referred to above are schematics, and are not necessarily exact.

### <Supplement>

(1) One aspect of the present invention is an ultrasound signal processing device that performs multiple transmission events of transmitting non-focused ultrasound beams to a subject by using an ultrasound probe having multiple transducers, that performs, for each of the transmission events, reception of ultrasound reflection from the subject and generation of an acoustic line signal based on the ultrasound reflection, and that combines acoustic line signals for the respective transmission events to generate a combined acoustic line signal, the ultrasound signal processing device including ultrasound signal processing circuitry configured to operate as: a transmitter that varies ultrasound beam travel direction between transmission events and performs each transmission event by causing the ultrasound probe to transmit an ultrasound beam traveling to an inside of the subject; a reception processor that generates an acoustic line signal for each transmission event that is performed by generating reception signal sequences for transducers based on ultrasound reflection that the ultrasound probe receives from a target region inside the subject and performing delay-and-summing using the reception signal sequences; a combiner that generates an intermediate combined acoustic line signal by combining, based on measurement points in the target region, acoustic line signals corresponding to the first to latest transmission events performed for a current combined acoustic line signal; an evaluator that judges whether a subsequent transmission event is to be performed for the current combined acoustic line signal by calculating an evaluation value from an energy value of the intermediate combined acoustic line signal and judging whether the evaluation value satisfies a predetermined condition; and an outputter that outputs the intermediate combined acoustic line signal as the current combined acoustic line signal once the evaluator judges that a subsequent transmission event is not to be performed for the current combined acoustic line signal.

Another aspect of the present invention is an ultrasound signal processing method of (i) performing multiple transmission events of transmitting non-focused ultrasound beams to a subject by using an ultrasound probe having multiple transducers, (ii) performing, for each of the transmission events, reception of ultrasound reflection from the subject and generation of an acoustic line signal based on the ultrasound reflection, and (iii) combining acoustic line signals for the respective transmission events to generate a combined acoustic line signal, the ultrasound signal processing method including: varying ultrasound beam travel direction between transmission events and performing each transmission event by causing the ultrasound probe to transmit an ultrasound beam traveling to an inside of the subject; generating an acoustic line signal for each transmission event that is performed by generating reception signal sequences for transducers based on ultrasound reflection that the ultrasound probe receives from a target region inside the subject and performing delay-and-summing using the reception signal sequences; generating an intermediate combined acoustic line signal by combining, based on measurement points in the target region, acoustic line signals corresponding to the first to latest transmission events performed for a current combined acoustic line signal; judging whether a subsequent transmission event is to be performed for the current combined acoustic line signal by calculating an evaluation value from an energy value of the intermediate combined acoustic line signal and judging whether the evaluation value satisfies a predetermined condition; and outputting the intermediate combined acoustic line signal as the current combined acoustic line signal once a judgment is made that a subsequent transmission event is not to be performed for the current combined acoustic line signal.

According to the present disclosure, the above-described structure enables adaptively changing the number of transmission events performed based on a relation between the number of acoustic line signals that are combined and the quality of the combined acoustic line signal. As such, both high spatial resolution and high temporal resolution can be achieved by suppressing the number of transmission events performed to as small as possible while keeping spatial resolution at a certain level or higher.

(2) The ultrasound signal processing device of (1), wherein ultrasound beam travel directions for two successive transmission events may extend to opposite sides of a normal line direction relative to a direction in which the transducers form a line.

The above-described structure prevents spatial biasing of ultrasound beam travel directions to one side along the direction in which the transducers form a line, regardless of the number of transmission events having been performed at the point when the evaluator makes the judgment that a subsequent transmission event is not to be performed. Thus, the above-described structure prevents a situation where the number of ultrasound beams having been transmitted is not the same at both sides of a normal line direction relative to the direction in which the transducers form a line. As such, the above-described structure prevents ununiformity of spatial resolution of the combined acoustic line signal in the direction in which the transducers form a line.

(3) The ultrasound signal processing device of (1) or (2), wherein when defining a transmission angle for a transmission event as an angle between an ultrasound beam travel direction for the transmission event and a normal line direction relative to a direction in which the transducers form a line, transmission angles for two successive transmission events may be such that (i) an absolute value of the transmission angle for the later transmission event is greater than an absolute value of the transmission angle for the earlier transmission event, or (ii) the absolute value of the transmission angle for the later transmission is equal to the absolute value of the transmission angle for the earlier transmission event.

This structure enables reducing the number of transmission events already performed at the point when the evaluator makes the judgment that a subsequent transmission event is not to be performed, with acoustic line signals with greater likelihood of improving intermediate combined acoustic line signal spatial resolution being used earlier in the combining of acoustic line signals.

(4) The ultrasound signal processing device of (1) through (3), wherein when defining a transmission angle for a transmission event as an angle between a ultrasound beam travel direction for the transmission event and a normal line direction relative to a direction in which the transducers form a line, the ultrasound signal processing circuitry may further operate as a transmission angle storage that stores a transmission angle sequence that defines transmission angles each corresponding to one transmission event.

(5) The ultrasound signal processing device of (4), wherein the transmission angle storage may store two or more transmission angle sequences, and the transmitter may select one of the transmission angle sequences according to at least one of: a cross-sectional area of the target region; a position of the target region in the subject; and tissue type at the target region.

The above-described structure enables using a suitable transmission angle sequence based on characteristics of the target region.

(6) The ultrasound signal processing device (1) through (5), wherein when defining a transmission angle for a transmission event as an angle between a ultrasound beam travel direction for the transmission event and a normal line direction relative to a direction in which the transducers form a line, the evaluator may calculate the evaluation value by normalizing an amount of change in the energy value of the intermediate combined acoustic line signal by using an energy value of an acoustic line signal corresponding to a 0° transmission angle, and the predetermined condition may be that the evaluation value is lower than a predetermined threshold.

The above-described structure enables detecting, based on change in energy, whether intermediate combined acoustic line signal spatial resolution improves through the combining of acoustic line signals.

(7) The ultrasound signal processing device of (1) through (6), wherein the evaluator may calculate the energy value of the intermediate combined acoustic line signal by summing amplitude values of the intermediate combined acoustic line signal within a predetermined region of the target region.

(8) The ultrasound signal processing device of (1) through (6), wherein the evaluator may convert the intermediate combined acoustic line signal into Fourier data by performing two-dimensional Fourier transform for a predetermined region of the target region, calculate total power of a spectrum of the Fourier data, and use the total power as the energy value of the intermediate combined acoustic line signal.

(9) The ultrasound signal processing device of (1) through (6), wherein the evaluator may convert data of the intermediate combined acoustic line signal for a predetermined region of the target region into an one-dimensional acoustic line signal for a depth direction, perform one-dimensional Fourier transform in the depth direction on the one-dimensional acoustic line signal to acquire Fourier data, calculate total power of a spectrum of the Fourier data, and use the total power as the energy value of the intermediate combined acoustic line signal.

The above-described structures enable estimating intermediate combined acoustic line signal spatial resolution by using a total of energy of the intermediate combined acoustic line signal.

(10) The ultrasound signal processing device of (8) or (9), wherein the evaluator may use the entire frequency range of the spectrum of the Fourier data to calculate the energy value of the intermediate combined acoustic line signal.

(11) The ultrasound signal processing device of (8) or (9), wherein the evaluator may use a predetermined frequency range of the spectrum of the Fourier data to calculate the energy value of the intermediate combined acoustic line signal.

The above-described structures enable estimating intermediate combined acoustic line signal spatial resolution based on specific frequency components of the intermediate combined acoustic line signal.

(12) The ultrasound signal processing device of (7) through (11), wherein the predetermined region may be the entire target region.

(13) The ultrasound signal processing device of (7) through (11), wherein the predetermined region may be a region of interest in the target region.

The above-described structures enable estimating intermediate combined acoustic line signal spatial resolution based on the entire target region or a specific part of the target region.

(14) The ultrasound signal processing device of (1) through (13), wherein once the evaluator judges that a subsequent transmission event is not to be performed for the current combined acoustic line signal, the outputter may vary a time point at which a transmission event for a subsequent combined acoustic line signal is to be commenced based on the number of transmission events performed to generate the current combined acoustic line signal.

The above-described structure enables improving frame rate, which is the frequency at which combined acoustic line signals are output, in accordance with the number of transmission events performed.

(15) The ultrasound signal processing device of (1) through (14), wherein the ultrasound signal processing circuitry may further operate as: a controller that holds therein a transmission count indicating the number of transmission events performed to generate the current combined acoustic line signal, once the evaluator judges that a subsequent transmission event is not to be performed for the current combined acoustic line signal, and in generation of each of one or more subsequent combined acoustic line signals, the controller may cause the transmitter to perform transmission of ultrasound beams the number of times indicated by the transmission count, and cause the outputter to output an acoustic line signal generated by combining all acoustic line signals corresponding to the ultrasound beams that the transmitter has transmitted as a combined acoustic line signal, regardless of the judgment by the evaluator.

With the above-described structure, the evaluator performs the setting of the number of transmission events only in the generation of some combined acoustic line signals, and in the generation of combined acoustic line signals subsequent to each of such combined acoustic line signals, the evaluator does not perform the judgment.

(16) The ultrasound signal processing device of (15), wherein in generation of each of a predetermined number of combined acoustic line signals performed following the holding of the transmission count, the controller may cause the transmitter to perform transmission of ultrasound beams the number of times indicated by the transmission count, and cause the outputter to output an acoustic line signal generated by combining all acoustic line signals corresponding to the ultrasound beams that the transmitter has transmitted as a combined acoustic line signal, regardless of the judgment by the evaluator, and in generation of a combined acoustic line signal subsequent to the predetermined number of combined acoustic line signals, the controller may cause the evaluator to perform the judgment of whether a subsequent transmission event is to be performed for the current combined acoustic line signal and cause the outputter to output an intermediate acoustic line signal as the current combined acoustic line signal once the evaluator judges that a subsequent transmission event is not to be performed for the current combined acoustic line signal.

With the above-described structure, the setting of the number of transmission events is performed at a regular basis.

(17) The ultrasound signal processing device of (15) or (16) further including an input receiver that receives user input, wherein the controller may cause the evaluator to perform the judgment of whether a subsequent transmission event is to be performed for a current combined acoustic line signal additionally when the input receiver receives a user instruction to determine the number of transmissions performed to generate the current combined acoustic line signal.

With the above-described structure, the setting of the number of transmission events is performed in accordance with a user instruction.

(18) The ultrasound signal processing device of (1) through (17), wherein the transmitter may transmit plane wave ultrasound beams each with a wavefront perpendicular to the ultrasound beam travel direction.

The above-described structure enables applying the present invention to synthesis of plane waves.

The ultrasound signal processing device, the ultrasound diagnostic device, and the ultrasound signal processing method pertaining to the present disclosure are useful for image diagnosis using ultrasound. In particular, the ultrasound signal processing device, the ultrasound diagnostic device, and the ultrasound signal processing method pertaining to the present disclosure make it possible to achieve both high spatial resolution and high temporal resolution, and have high industrial applicability in the field of medical diagnostic equipment and the like.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

What is claimed is:

1. An ultrasound signal processing device that performs multiple transmission events of transmitting non-focused ultrasound beams to a subject by using an ultrasound probe having multiple transducers, that performs, for each of the transmission events, reception of ultrasound reflection from the subject and generation of an acoustic line signal based on the ultrasound reflection, and that combines acoustic line signals for the respective transmission events to generate a combined acoustic line signal,
the ultrasound signal processing device comprising ultrasound signal processing circuitry configured to operate as:
a transmitter that varies ultrasound beam travel direction between transmission events and performs each transmission event by causing the ultrasound probe to transmit an ultrasound beam traveling to an inside of the subject;
a reception processor that generates an acoustic line signal for each transmission event that is performed by generating reception signal sequences for transducers based on ultrasound reflection that the ultrasound probe receives from a target region inside the subject and performing delay-and-summing using the reception signal sequences;
a combiner that generates an intermediate combined acoustic line signal by combining, based on measurement points in the target region, acoustic line signals corresponding to the first to latest transmission events performed for a current combined acoustic line signal;
an evaluator that judges whether a subsequent transmission event is to be performed for the current combined acoustic line signal by calculating an evaluation value from an energy value of the intermediate combined acoustic line signal and judging whether the evaluation value satisfies a predetermined condition; and
an outputter that outputs the intermediate combined acoustic line signal as the current combined acoustic line signal once the evaluator judges that a subsequent transmission event is not to be performed for the current combined acoustic line signal.

2. The ultrasound signal processing device of claim 1, wherein
ultrasound beam travel directions for two successive transmission events extend to opposite sides of a normal line direction relative to a direction in which the transducers form a line.

3. The ultrasound signal processing device of claim 1, wherein
when defining a transmission angle for a transmission event as an angle between an ultrasound beam travel direction for the transmission event and a normal line direction relative to a direction in which the transducers form a line,
transmission angles for two successive transmission events are such that (i) an absolute value of the transmission angle for the later transmission event is greater than an absolute value of the transmission angle for the earlier transmission event, or (ii) the absolute value of the transmission angle for the later transmission is equal to the absolute value of the transmission angle for the earlier transmission event.

4. The ultrasound signal processing device of claim 1, wherein
when defining a transmission angle for a transmission event as an angle between a ultrasound beam travel direction for the transmission event and a normal line direction relative to a direction in which the transducers form a line,
the ultrasound signal processing circuitry further operates as a transmission angle storage that stores a transmission angle sequence that defines transmission angles each corresponding to one transmission event.

5. The ultrasound signal processing device of claim 4, wherein
the transmission angle storage stores two or more transmission angle sequences, and
the transmitter selects one of the transmission angle sequences according to at least one of: a cross-sectional area of the target region; a position of the target region in the subject; and tissue type at the target region.

6. The ultrasound signal processing device of claim 1, wherein
when defining a transmission angle for a transmission event as an angle between a ultrasound beam travel direction for the transmission event and a normal line direction relative to a direction in which the transducers form a line,
the evaluator calculates the evaluation value by normalizing an amount of change in the energy value of the intermediate combined acoustic line signal by using an energy value of an acoustic line signal corresponding to a 0° transmission angle, and
the predetermined condition is that the evaluation value is lower than a predetermined threshold.

7. The ultrasound signal processing device of claim 1, wherein
the evaluator calculates the energy value of the intermediate combined acoustic line signal by summing amplitude values of the intermediate combined acoustic line signal within a predetermined region of the target region.

8. The ultrasound signal processing device of claim 1, wherein
the evaluator converts the intermediate combined acoustic line signal into Fourier data by performing two-dimensional Fourier transform for a predetermined region of the target region, calculates total power of a spectrum of the Fourier data, and uses the total power as the energy value of the intermediate combined acoustic line signal.

9. The ultrasound signal processing device of claim 1, wherein
the evaluator converts data of the intermediate combined acoustic line signal for a predetermined region of the target region into an one-dimensional acoustic line signal for a depth direction, performs one-dimensional Fourier transform in the depth direction on the one-dimensional acoustic line signal to acquire Fourier data, calculates total power of a spectrum of the Fourier data, and uses the total power as the energy value of the intermediate combined acoustic line signal.

10. The ultrasound signal processing device of claim 8, wherein
the evaluator uses the entire frequency range of the spectrum of the Fourier data to calculate the energy value of the intermediate combined acoustic line signal.

11. The ultrasound signal processing device of claim 8, wherein
the evaluator uses a predetermined frequency range of the spectrum of the Fourier data to calculate the energy value of the intermediate combined acoustic line signal.

12. The ultrasound signal processing device of claim 7, wherein
the predetermined region is the entire target region.

13. The ultrasound signal processing device of claim 7, wherein
the predetermined region is a region of interest in the target region.

14. The ultrasound signal processing device of claim 1, wherein
once the evaluator judges that a subsequent transmission event is not to be performed for the current combined acoustic line signal, the outputter varies a time point at which a transmission event for a subsequent combined acoustic line signal is to be commenced based on the number of transmission events performed to generate the current combined acoustic line signal.

15. The ultrasound signal processing device of claim 1, wherein
the ultrasound signal processing circuitry further operates as:
a controller that holds therein a transmission count indicating the number of transmission events performed to generate the current combined acoustic line signal, once the evaluator judges that a subsequent transmission event is not to be performed for the current combined acoustic line signal, and
in generation of each of one or more subsequent combined acoustic line signals, the controller causes the transmitter to perform transmission of ultrasound beams the number of times indicated by the transmission count, and causes the outputter to output an acoustic line signal generated by combining all acoustic line signals corresponding to the ultrasound beams that the transmitter has transmitted as a combined acoustic line signal, regardless of the judgment by the evaluator.

16. The ultrasound signal processing device of claim 15, wherein
in generation of each of a predetermined number of combined acoustic line signals performed following the holding of the transmission count, the controller causes the transmitter to perform transmission of ultrasound beams the number of times indicated by the transmission count, and causes the outputter to output an acoustic line signal generated by combining all acoustic line signals corresponding to the ultrasound beams that the transmitter has transmitted as a combined acoustic line signal, regardless of the judgment by the evaluator, and
in generation of a combined acoustic line signal subsequent to the predetermined number of combined acoustic line signals, the controller causes the evaluator to perform the judgment of whether a subsequent transmission event is to be performed for the current combined acoustic line signal and causes the outputter to output an intermediate acoustic line signal as the current combined acoustic line signal once the evaluator judges that a subsequent transmission event is not to be performed for the current combined acoustic line signal.

17. The ultrasound signal processing device of claim 15 further comprising
an input receiver that receives user input, wherein
the controller causes the evaluator to perform the judgment of whether a subsequent transmission event is to be performed for a current combined acoustic line signal additionally when the input receiver receives a user instruction to determine the number of transmissions performed to generate the current combined acoustic line signal.

18. The ultrasound signal processing device of claim 1, wherein
the transmitter transmits plane wave ultrasound beams each with a wavefront perpendicular to the ultrasound beam travel direction.

19. An ultrasound diagnostic device comprising:
an ultrasound probe; and
an ultrasound signal processing device that performs multiple transmission events of transmitting non-focused ultrasound beams to a subject by using an ultrasound probe having multiple transducers, that performs, for each of the transmission events, reception of ultrasound reflection from the subject and generation of an acoustic line signal based on the ultrasound reflection, and that combines acoustic line signals for the respective transmission events to generate a combined acoustic line signal,
the ultrasound signal processing device comprising ultrasound signal processing circuitry configured to operate as:
a transmitter that varies ultrasound beam travel direction between transmission events and performs each transmission event by causing the ultrasound probe to transmit an ultrasound beam traveling to an inside of the subject;
a reception processor that generates an acoustic line signal for each transmission event that is performed by generating reception signal sequences for transducers based on ultrasound reflection that the ultrasound probe receives from a target region inside the subject and performing delay-and-summing using the reception signal sequences;
a combiner that generates an intermediate combined acoustic line signal by combining, based on measurement points in the target region, acoustic line signals corresponding to the first to latest transmission events performed for a current combined acoustic line signal;
an evaluator that judges whether a subsequent transmission event is to be performed for the current combined acoustic line signal by calculating an evaluation value from an energy value of the intermediate combined acoustic line signal and judging whether the evaluation value satisfies a predetermined condition; and
an outputter that outputs the intermediate combined acoustic line signal as the current combined acoustic line signal once the evaluator judges that a subsequent transmission event is not to be performed for the current combined acoustic line signal.

20. An ultrasound signal processing method of (i) performing multiple transmission events of transmitting non-focused ultrasound beams to a subject by using an ultrasound probe having multiple transducers, (ii) performing, for each of the transmission events, reception of ultrasound reflection from the subject and generation of an acoustic line signal based on the ultrasound reflection, and (iii) combining acoustic line signals for the respective transmission events to generate a combined acoustic line signal, the ultrasound signal processing method comprising:
varying ultrasound beam travel direction between transmission events and performing each transmission event by causing the ultrasound probe to transmit an ultrasound beam traveling to an inside of the subject;
generating an acoustic line signal for each transmission event that is performed by generating reception signal sequences for transducers based on ultrasound reflection that the ultrasound probe receives from a target region inside the subject and performing delay-and-summing using the reception signal sequences;
generating an intermediate combined acoustic line signal by combining, based on measurement points in the target region, acoustic line signals corresponding to the first to latest transmission events performed for a current combined acoustic line signal;
judging whether a subsequent transmission event is to be performed for the current combined acoustic line signal by calculating an evaluation value from an energy value of the intermediate combined acoustic line signal and judging whether the evaluation value satisfies a predetermined condition; and
outputting the intermediate combined acoustic line signal as the current combined acoustic line signal once a judgment is made that a subsequent transmission event is not to be performed for the current combined acoustic line signal.
